(19) United States
(12) Patent Application Publication
(10) Pub. No.: US 2023/0410297 A1
(43) Pub. Date: Dec. 21, 2023

(54) CONTROL DEVICE, CONTROL METHOD, AND RECORDING MEDIUM

(71) Applicant: KONICA MINOLTA, INC., Tokyo (JP)

(72) Inventors: Atsushi TANEDA, Tokyo (JP); Akira HIROSHIGE, Tokyo (JP); Naoki HAYASHI, Tokyo (JP)

(73) Assignee: KONICA MINOLTA, INC., Tokyo (JP)

(21) Appl. No.: 18/200,072

(22) Filed: May 22, 2023

(30) Foreign Application Priority Data
Jun. 17, 2022 (JP) 2022-098142
Jun. 17, 2022 (JP) 2022-098146
Jun. 17, 2022 (JP) 2022-098148

(51) Int. Cl. G06T 7/00

(52) U.S. Cl. CPC G06T 7/0012; G06T 2207/10116

(57) ABSTRACT

A control device includes a hardware processor that obtains a plurality of radiographs satisfying a predetermined condition, and identifies a radiograph to be subjected to a predetermined process, from among the plurality of radiographs obtained.

### CROSS-REFERENCE TO RELATED APPLICATIONS

The entire disclosure of Japanese Patent Application No. 2022-098148, filed on Jun. 17, 2022, Japanese Patent Application No. 2022-098142, filed on Jun. 17, 2022, and Japanese Patent Application No. 2022-098146, filed on Jun. 17, 2022, is incorporated herein by reference in its entirety.

### TECHNICAL FIELD

The present invention relates to a control device, a control method, and a recording medium.

### DESCRIPTION OF THE RELATED ART

Conventionally, in a medical field, a predetermined process is executed to an image taken by a radiographic imaging system. For example, JP 2016-87279A describes that information indicating an elapsed time from a reference date and time to a radiography date and time is assigned to a taken image, based on information about radiography of a test subject.

Incidentally, radiographing requires a series of radiographing tasks from positioning to imaging, to image confirmation after radiographing, and to execution of the predetermined process.

A technologist performing radiography sometimes performs a large number of radiographing tasks a day. In radiographing, also to reduce the physical burden of a test subject, it is preferable to release the test subject as soon as possible. Accordingly, the technologist is required to execute and complete the radiographing tasks in a short time period.

The taken image is required to be an image easily viewable for a clinician and a radiologist. It is important to appropriately execute predetermined processes, which are called post-processes, to the taken image.

### SUMMARY OF THE INVENTION

However, in a limited time period in a day, it is difficult to execute both a large number of radiographing operations and the appropriate predetermined process for achieving an image facilitating diagnosis at the same time. The burden on the technologist is large, and patients and hospital management are also burdened: the waiting time period increases, for instance.

The invention in JP 2016-87279A assigns complementary information, which is the elapsed time period, to follow-up. However, it only assigns a piece of information in a specific case. An improvement of effectively advancing the processing for a large number of tests occurring a day cannot be expected. Thus, the reduction in load is insufficient.

The present invention has an object to provide a control device, a control method, and a program that can effectively execute a task for executing the predetermined processes for a radiograph.

To achieve at least one of the abovementioned objects, according to an aspect of the present invention, a control device reflecting one aspect of the present invention includes a hardware processor that
obtains a plurality of radiographs satisfying a predetermined condition, and
identifies a radiograph to be subjected to a predetermined process, from among the plurality of obtained radiographs.

To achieve at least one of the abovementioned objects, according to an aspect of the present invention, a control method reflecting one aspect of the present invention includes:
obtaining a plurality of radiographs satisfying a predetermined condition; and
identifying a radiograph to be subjected to a predetermined process, from among the plurality of radiographs obtained by the obtaining.

To achieve at least one of the abovementioned objects, according to an aspect of the present invention, a recording medium reflecting one aspect of the present invention is
a non-transitory computer readable recording medium storing a program causing a computer used for a control device to:
obtain a plurality of radiographs satisfying a predetermined condition, and
identify a radiograph to be subjected to a predetermined process, from among the plurality of obtained radiographs.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 shows an X-ray radiographic system according to one embodiment of the present invention;

FIG. 2 shows a configuration of a radiography control device;

FIG. 3 is a flowchart showing the flow of a radiographing process;

FIG. 4 shows an example of a test screen of the radiography control device;

FIG. 5 is a flowchart showing the flow of a post-process determination process;

FIG. 6 shows an example of a test screen of the radiography control device;

FIG. 7 shows an example of a test screen of the radiography control device;

FIG. 8 shows an example of a test screen of the radiography control device;

FIG. 9A shows an example of a test screen of the radiography control device;

FIG. 9B shows an example of a test screen of the radiography control device;

FIG. 10 shows an example of a test screen of the radiography control device;

FIG. 11 shows an example of a test screen of the radiography control device;

FIG. 12 shows comparison between the post-processes this time and the last-time post-processes;

FIG. 13 shows the content of the post-process this time;

FIG. 14 shows an example of a test screen of the radiography control device;

FIG. 15 is a flowchart showing the flow of a post-process conclusion process;

FIG. 16 shows an example of post-process types with respect to specific radiography sites and specific cases;

FIG. 17 shows an example of a test screen of the radiography control device;

FIG. 18 shows an example of a test screen of the radiography control device;

FIG. 19 is a flowchart showing the flow of the radiographing process;

FIG. 20 shows an example of a test screen of the radiography control device; and

FIG. 21 shows an example of a test screen of the radiography control device.

### DETAILED DESCRIPTION

Hereinafter, one or more embodiments of the present invention will be described with reference to the drawings. However, the scope of the invention is not limited to the disclosed embodiments.

### First Embodiment

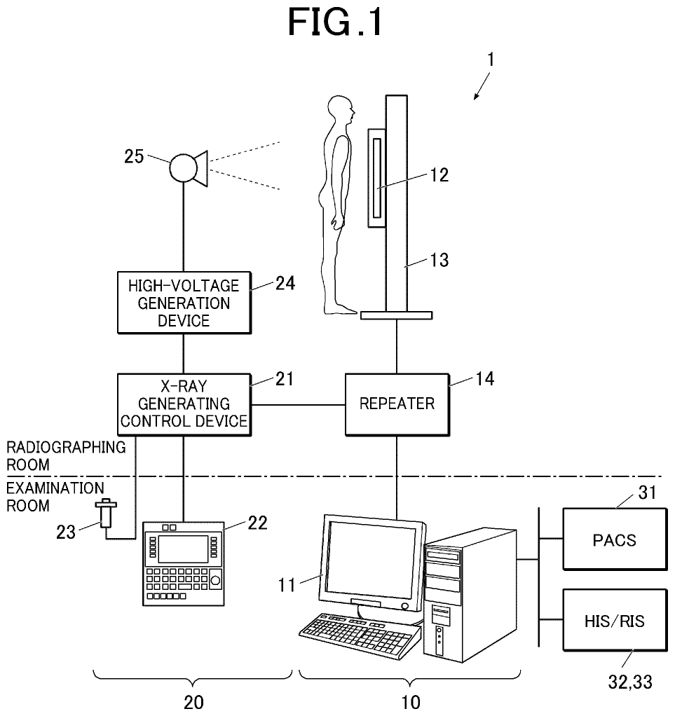

FIG. 1 shows an X-ray radiographic system 1 according to one embodiment of the present invention.

The X-ray radiographic system 1 is an integrated radiographic system that exchanges a signal and the like between an X-ray generation apparatus 20 and an X-ray radiographic apparatus 10, and performs X-ray (radiation) radiographing (hereinafter, represented as radiographing) while causing both the apparatuses to cooperate with each other.

### (Configuration of X-Ray Radiographic System)

As shown in FIG. 1, the X-ray radiographic system 1 includes the X-ray radiographic apparatus 10, and the X-ray generation apparatus 20.

The X-ray radiographic system 1 is connected to picture archiving and communication systems (PACS) 31, hospital information systems (HIS) 32, and radiology information systems (RIS) 33 through a communication network.

In the communication network that includes the X-ray radiographic system 1, the PACS 31, the HIS 32, and the RIS 33, information is transmitted and received in conformity with the DICOM (Digital Image and Communications in Medicine) standard, for example.

The X-ray radiographic apparatus 10 includes a radiography control device 11 serving as a control device, an FPD (Flat Panel Display) 12, a radiographic stand 13, and a repeater 14.

For example, the X-ray radiographic apparatus 10 visualizes X-rays having been transmitted through a radiographing target site (hereinafter, represented as a radiography site), such as a chest and an abdomen, thus taking an X-ray image (hereinafter, represented as an image) indicating the state in the body.

The FPD 12 is an image pickup device that detects X-rays having been emitted from an X-ray tube device and transmitted through a test subject, and outputs image data.

For example, the FPD 12 is attached to the radiographic stand 13, and is communicably connected to the radiography control device 11 via the radiographic stand 13 and the repeater 14 by wired communication.

The FPD 12 may be connected to the radiography control device 11 by wireless communication. In a case where the FPD 12 has a wireless communication function, the FPD 12 is not necessarily attached to the dedicated radiographic stand 13, and may be placed on a bed where the test subject is lying on their back, or be held by the test subject themselves, and thus be used.

The FPD 12 includes, for example, a scintillator that converts incident X-rays into light, PDs (Photo Diodes) arranged in a matrix corresponding to pixels, and TFT (Thin Film Transistor) switches arranged corresponding to the respective PDs (these are not shown).

The incident X-rays are converted by the scintillator into light, which enters into the PDs and is accumulated as charges on a pixel-by-pixel basis. The charges accumulated in the PDs flow out through the TFT switches and signal lines, are amplified and A/D-converted, and output as image data to the radiography control device 11.

Note that the FPD 12 may be of an indirect conversion type described above, or a direct conversion type that directly converts X-rays into an electric signal.

The radiographic stand 13 detachably holds the FPD 12 so that an X-ray incident surface of the FPD 12 can have an attitude facing the X-my tube device 25. FIG. 1 exemplifies, as the radiographic stand 13, an upright-position radiographic stand that radiographs a test subject at an upright attitude.

The radiographic stand 13 may be a decubitus-position radiographic stand that radiographs a test subject at a decubitus position.

For example, the radiographic stand 13 is communicably connected to the radiography control device 11 via the repeater 14 by wired communication.

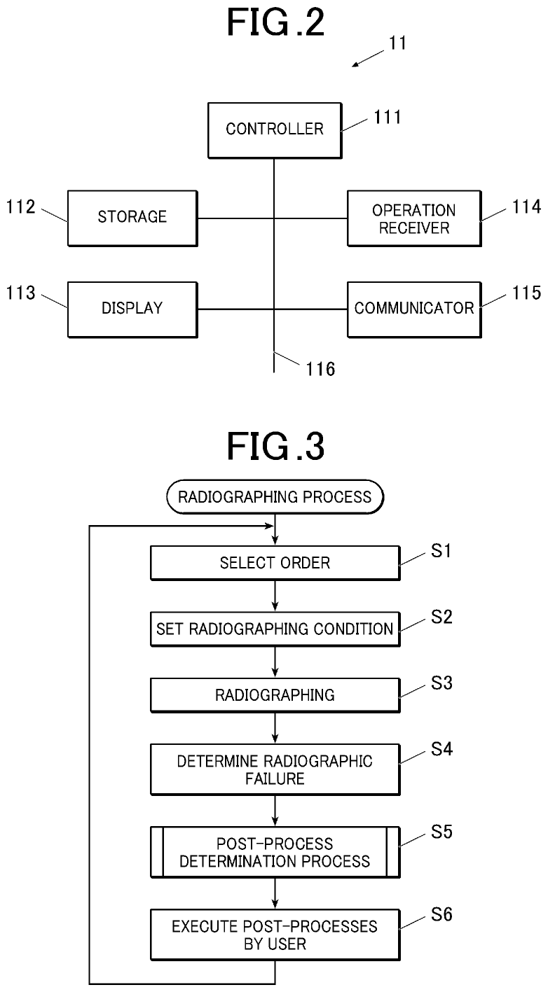

The radiography control device 11 cooperates with an X-ray generating control device 21, and controls the X-ray radiographic system 1. For example, the radiography control device 11 transmits a detection condition to the FPD 12, and configures the setting. The detection condition includes a radiographing image size, a frame rate (in a case of dynamic radiographing), and information about signal processing executed by the FPD 12 (e.g., the gain of an amplifier etc.). The radiography control device 11 controls each operation of the FPD 12, obtains image data from the FPD 12, applies predetermined image processing to the data, and causes a display 113 (see FIG. 2) to display the data.

Note that the radiography control device 11 may constitute part of the X-ray generation apparatus 20. For example, the radiography control device 11 may include a function as an X-ray generation console 22 of the X-ray generation apparatus 20 (what is called an integrated X-ray radiographic system).

The X-ray radiographic system 1 may include a display terminal apparatus, not shown. The display terminal apparatus may display content identical to that displayed on the display 113, or a part of the content displayed on the display 113. The display terminal apparatus may be capable of using the image processing and a part of the function of the X-ray generation console 22.

The X-ray generation apparatus 20 includes the X-ray generating control device 21, the X-ray generation console 22, an irradiation switch 23, a high-voltage generation device 24, and the X-ray tube device 25.

The X-ray tube device 25 is disposed opposite to the FPD 12, with the test subject intervening between them. A high voltage is applied by the high-voltage generation device 24 to the X-ray tube device 25, which generates X-rays, and emits the X-rays toward the test subject. The X-ray tube device 25 includes an X-ray movable diaphragm that adjusts the X-ray irradiation field.

The X-ray generation console 22, and the irradiation switch 23 are connected to the X-ray generating control device 21 via a signal cable.

The X-ray generation console 22 is an operation console for receiving an input of irradiation parameters and the like. The irradiation switch 23 is a switch for issuing an instruction for X-ray irradiation, and is made up of a two-step automatic reset push button switch, for example. When a first-step press operation is performed on the irradiation switch 23, a warming up start signal for starting warming up of the X-ray tube device 25 is transmitted to the X-ray generating control device 21. When a second-step press operation is performed, an irradiation start signal for causing the X-ray tube device 25 to start X-ray irradiation is transmitted to the X-ray generating control device 21.

The X-ray generating control device 21 controls the operations of the high-voltage generation device 24 and the X-ray tube device 25, based on the irradiation parameters (radiation irradiation parameters) from the X-ray generation console 22 and on the control signals (the warming up start signal and the irradiation start signal) from the irradiation switch 23.

The irradiation parameters may be set through the X-ray generation console 22, and set using the radiography control device 11.

FIG. 2 shows the configuration of the radiography control device 11. As shown in FIG. 2, the radiography control device 11 includes the controller 111 (hardware processor), a storage 112, the display 113, an operation receiver 114, a communicator 115, and the like.

The controller 111 includes a CPU (Central Processing Unit), a ROM (Read Only Memory), a RAM (Random Access Memory), and the like (none is shown).

The ROM stores basic programs and basic setting data.

The CPU reads programs corresponding to the processing content from the ROM or the storage 112, loads the programs into the RAM, and executes the loaded programs, thus centrally controlling the operations of the FPD 12 and the like.

The controller 111 obtains a plurality of radiographs satisfying a predetermined condition. Here, the controller 111 functions as an obtainer.

The controller 111 identifies a radiograph to be subjected to post-processes (predetermined processes), from among the plurality of radiographs obtained by the obtainer. Here, the controller 111 functions as an identifier.

The controller 111 presents a display that discriminates the radiograph that is identified by the identifier to be subjected to the post-processes (predetermined processes), from the radiograph that is unidentified by the identifier. Here, the controller 111 functions as a display controller.

The controller 111 executes the post-processes (predetermined processes) to the radiograph that is identified by the identifier to be subjected to the post-processes (predetermined processes). Here, the controller 111 functions as a processor.

The controller 111 accepts the post-processes (predetermined processes) by the user to the radiograph that is identified by the identifier to be subjected to the post-processes (predetermined processes). Here, the controller 111 functions as an acceptor.

When the processor switches multiple radiographs and executes the post-processes (predetermined processes), the controller 111 assigns a second operation receiver (described later) a function of switching the radiograph to be displayed on the display 113.

The storage 112 may be an auxiliary storage device, such as an HDD (Hard Disk Drive) or an SSD (Solid State Drive), for example. The storage 112 may be a disk drive that drives an optical disk, such as a CD (Compact Disc) and a DVD (Digital Versatile Disc), or a magnet-optical disk, such as an MO (Magneto-Optical disk), and reads and writes information. For example, the storage 112 may be a memory card, such as a USB memory or an SD card.

The storage 112 stores various programs executed by the controller 111, parameters required to execute the programs, and data, such as processing results.

The storage 112 stores an image taken by the X-ray radiographic apparatus 10.

The storage 112 also stores test order information.

The test order information includes patient information (e.g., a patient ID, patient name, birth date, gender, location of the patient) on a patient as a test subject, a radiographing condition, test items (pulmonary ventilation, pulmonary perfusion, etc.), a test history of the test subject (the radiographing condition in the last test, etc.), and consultation information (a medical department, a doctor in attendance, a consultation comment, etc.).

The radiographing condition includes attitude information in radiographing (e.g., the attitude (upright position/decubitus position)), the irradiation direction (back/front/side), radiography site information (e.g., chest), the tube voltage and the tube current, the irradiation time period (mAs value), the frame rate (in the case of dynamic radiographing), the body-built of the test subject, presence or absence of a grid, and the like.

The display 113 is made up of, for example, a flat panel display, such as a liquid crystal display or an organic EL display.

The display 113 displays the content of the test order and a taken image, based on the display control signal from the controller 111.

The operation receiver 114 includes: a keyboard having cursor keys, numeric input keys, and various function keys; and a pointing device, such as a mouse.

The operation receiver 114 accepts an operation signal input through a key operation or a mouse operation, and outputs the signal to the controller 111.

Note that the display 113 and the operation receiver 114 may have an integral configuration, such as of a flat panel display with a touch panel, for example.

The communicator 115 is a communication interface, such as an NIC (Network Interface Card), a MODEM (Modulator-DEModulator), and a USB (Universal Serial Bus), for example.

The controller 111 transmits and receives various types of information to and from an apparatus connected to a network, such as a wired/wireless LAN, via the communicator 115, in conformity with the DICOM standard.

A communication interface for near field wireless communication, such as NFC (Near Field Communication) or Bluetooth® may be applied to the communicator 115.

### (Operation of X-Ray Radiographic System)

Next, a radiographing process in the X-ray radiographic system 1 shown in FIG. 3 is described.

Here, it is assumed that test orders are preliminarily registered in the radiography control device 11. The test order may be input from an external system, such as the HIS 32 or the RIS 33, or manually input by a radiographer as a user (a technologist, a radiologist, a diagnostician, etc.) through the operation receiver 114.

### (Radiographing Process)

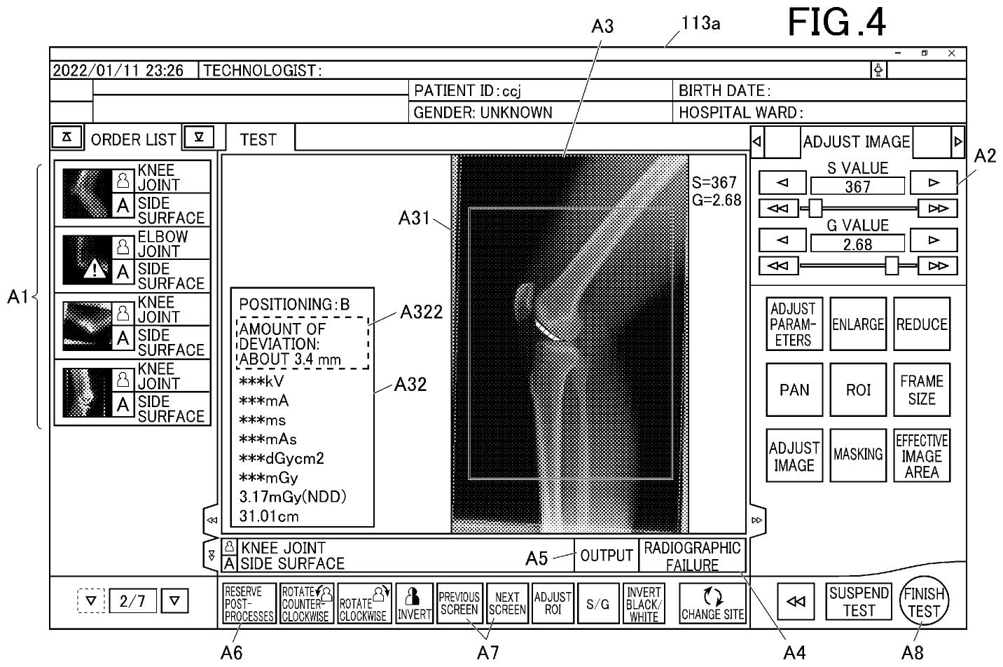

In a radiographing process, first, the controller 111 of the radiography control device 11 displays, on the display 113, a test screen 113a shown in FIG. 4, and accepts selection of the test order for execution of radiographing by the radiographer (Step S1). The radiographer selects the test order for execution of radiographing from among registered test orders by pressing a radiographing selection button A1.

FIG. 4 shows an example of the test screen 113a displayed on the display 113.

The test screen 113a is provided with not only the radiographing selection buttons A1 on each of which radiographing contents (the radiography site, radiographing direction, etc.) included in the test order information are displayed, but also a setting area A2 for image adjustment for selected radiographing, an image display area A3 for displaying a taken image, a radiographic failure button A4, an output button A5, a post-process reservation button A6 for designating an image as a post-process target, a switching button A7 for switching the image displayed in the image display area A3, a test finish button A8, and the like. Note that at the stage of Step S1, no image is displayed in the image display area A3.

When the test order is selected, the controller 111 transmits the radiographing condition included in the test order to the X-ray generating control device 21 and the FPD 12, and configures the setting (Step S2).

During this, the radiographer arranges the test subject between the X-ray tube device 25 and the radiographic stand 13, thus achieving positioning. Here, the positioning is setting of the body posture of the test subject during radiographing, for example. The radiographer instructs the test subject to take a certain respiratory status (deep respiration etc.).

Next, the controller 111 accepts a press operation on the irradiation switch 23 by the radiographer, and performs radiographing (Step S3).

In a case where an irradiation time period is set as in simple X-ray radiography, irradiation with X-rays is finished upon a lapse of the predetermined irradiation time period. In the case of dynamic radiographing, irradiation with X-rays is continuously performed in a time period in which the second-step press operation on the irradiation switch 23 is applied, and when the press operation on the irradiation switch 23 is released, the irradiation with X-rays is finished.

The taken image is transmitted from the FPD 12 to the radiography control device 11.

Here, it is assumed that the radiographer is in a state of having performed the radiographing multiple times. That is, the controller 111 obtains a plurality of radiographs taken by the same radiographer (radiographs satisfying the predetermined condition). Note that the plurality of radiographs satisfying the predetermined condition may be a plurality of radiographs in accordance with the test order, a plurality of radiographs taken by radiographing the same test subject, a plurality of radiographs taken on the same radiography date, a plurality of radiographs taken by radiographing the same radiography site, or a plurality of radiographs taken by radiographing in a predetermined time period. The plurality of radiographs satisfying the predetermined condition are only required to be a plurality of radiographs taken in one test. Across the plurality of radiographs, the radiographers, the radiography sites, and the medical departments may vary. Across the plurality of radiographs, a plurality of conditions among the same test subject, the same radiography date, the same radiography site, the same radiographer, and radiographing in the predetermined time period may be satisfied. The plurality of radiographs satisfying the predetermined condition may be a plurality of frame images taken by one time of dynamic radiographing. The plurality of radiographs satisfying the predetermined condition may be a plurality of frame images taken by multiple times of dynamic radiographing in one test.

As shown in FIG. 4, the controller 111 then displays a taken image A31, and the radiographing condition A32 in the radiographing, in the image display area A3.

Next, the controller 111 accepts determination of whether or not the image displayed in the image display area A3 is regarded as a radiographic failure by the radiographer (radiographic failure determination) (Step S4). To regard it as a radiographic failure, the radiographer presses the radiographic failure button A4.

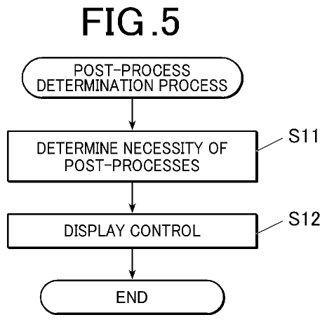

Next, the controller 111 determines whether or not post-processes as predetermined processes to be applied to the image are required (a post-process determination process shown in FIG. 5) (Step S5).

The post-processes include at least one of ROI (Region Of Interest) adjustment, effective image area setting, S/G value adjustment, rotation/inversion/rotate by any angle, image processing (E process/F process/H process/scattered radiation correction/image processing condition changing, etc.), emphasizing processes (a catheter distal end emphasizing process as a frequency emphasizing process, gauze emphasizing process/other emphasizing processes, etc.), and a grid removal process, masking, trimming, marker/stump/overlay, and output setting.

### (Post-Process Determination Process)

In the post-process determination process, the controller 111 determines whether or not the post-processes are required to be applied to the image taken in Step S3 (taken this time), and stores the determination result in the storage 112 (Step S11). That is, the controller 111 identifies a radiograph to be subjected to predetermined processes (post-processes), from among the plurality of radiographs obtained by the obtainer. In the case where radiography this time is dynamic radiography, the controller 111 may perform the determination in units of frame images, or collectively perform the determination of the plurality of frame images in one time of radiography.

Specifically, the controller 111 compares the image taken this time with an image that is a preset comparison target, and determines the necessity of the post-processes, based on the comparison result.

For example, if the comparison target image is the last-time image taken by radiographing the same radiography site as the radiography site radiographed this time, and there is no difference in the radiographing condition, alignment, resolution or the like between the image taken this time and the image taken last time, the controller 111 determines that the post-processes are not required.

If the image taken this time is compared with the image taken last time by radiographing the same radiography site, and the difference described above is present, the controller 111 displays, in the image display area A3, a comparison result A322 indicating the difference as information for indicating the necessity of the post-processes, or as complementary information for performing confirmation and adjustment in a focused manner for the post-processes, as shown in FIG. 4. Accordingly, the comparison result A322 may indicate not only the difference in coordinates of a specific structural object, but also a correction guide value indicating how the deviation is corrected with reference to an allowable threshold of difference.

The controller 111 determines the necessity of the post-processes to the image taken this time, based on the post-process applied to the preset comparison target image.

For example, if the comparison target is the image taken last time by radiographing the same radiography site as the radiography site radiographed this time, and the post-processes applied to the image taken last time are only predetermined fixed processes, the controller 111 automatically applies the same fixed processes to the image taken this time, and determines that the other post-processes are not required. The fixed processes are preset basic processes. Specifically, the processes include assignment of an annotation indicating a left and right direction, and the necessity of applying a scattered radiation correction process in accordance with use of the grid during radiography. Furthermore, since in a follow-up case it is often intended to interpret a radiograph at the same size as the size of a previous image, only an output process for the sake of archiving is performed, while negating the need of automatic adjustment by trimming and of an image processing system in a specific modality.

If the image taken this time satisfies a specific condition, the controller 111 determines that the post-processes to the image taken this time are not required. The specific condition is a specific radiography site, specific usage, a respiratory cycle, etc.

For example, if the radiography site of the image taken this time is a chest front/side that is frequently radiographed in general radiography, the image processing condition is often fixed and routinized. Accordingly, the controller 111 determines that the post-processes are not required. In a case where the radiography site is an orthopedic joint system, such as a knee joint, the viewability of the region of interest often varies depending on the irradiation direction and the angle of the subject, and the viewability is often supported by the post-processes. Accordingly, it is determined that the post-processes are required. In a case where the radiography site is a chest front/side, and the radiographing is requested by a specific medical department or a specific doctor, the degree of gradation process may be concerned on a clinician-by-clinician basis, and the post-processes can be determined to be required. The determination is made based on the radiography site as an example. However, the necessity of the post-processes may be determined by a combination with another condition.

For example, in a case where the image taken this time is used for a surgical operation as specific usage, the taken image is immediately confirmed. Accordingly, the controller 111 determines that the post-processes to the image taken this time is not required.

The controller 111 determines the necessity of the post-processes to the image taken this time, based on an input operation by the user. Here, the user may be a technologist, a radiologist, a diagnostician or the like, other than the radiographer.

Specifically, the controller 111 accepts determination by the user about whether or not to apply the post-processes to the image taken this time, through a post-process reservation button A6. If the user determines to apply the post-processes to the image taken this time, they press the post-process reservation button A6. When the post-process reservation button A6 is pressed, the controller 111 determines that the post-processes are required.

The controller 111 also determines the necessity of the post-processes to the image taken this time, based on external information.

For example, a case is described where the X-ray radiographic system 1 includes an optical camera (not shown) that takes an optical image in a range including at least a part of the area irradiated with X-rays by the X-ray tube device 25. That is, the optical camera may photograph a range including the entire test subject and the X-ray radiographic apparatus 10, without limitation to the area irradiated with X-rays.

In this case, the controller 111 determines the radiographing direction of the image taken this time, based on an optical image that is external information, and applies a process of assigning, as an annotation, the radiographing direction determined for the image taken this time. The controller 111 then determines that the process is not required in the post-processes.

Note that for example, the controller 111 may detect a foreign body, such as gauze, based on the optical image that is external information. If a foreign body, such as gauze, is not detected, the controller 111 determines that the catheter distal end emphasizing process and the gauze emphasizing process are not required in the post-processes.

For example, the controller 111 may determine whether or not a grid is used for radiography this time, based on the optical image that is external information. If no grid is used, the controller 111 determines that the grid removal process is not required in the post-processes.

For example, the controller 111 determines the posture of the test subject during radiography, based on the optical image that is external information, and applies a process of assigning the posture of the test subject, as an annotation or a comment, to the image taken this time. The controller 111 determines that the process is not required in the post-processes.

For example, the controller 111 determines the posture of the test subject during radiography, based on the optical image that is external information, and determines the necessity of the post-processes, based on the test subject, and the X-ray irradiation position or the X-ray irradiation direction (e.g., irradiation from the back of the test subject, irradiation from the front of the test subject, etc.). For example, in a case where the radiography site is an ankle, the controller 111 determines the posture of the test subject about whether the test subject on the radiographic stand with the leg being in an upright state or in a laid state, and determines whether the posture of the test subject is the same as the posture taken during the last radiography. The controller 111 then determines the necessity of the post-processes, based on the determination result.

For example, the controller 111 determines whether radiography has been performed with the test subject in a sitting state or in a standing state, based on the optical image that is external information, and determines a state of load application to the leg of the test subject and the gravity direction, based on the determination result.

As described above, to determine the posture of the test subject during radiography, the controller 111 sometimes uses what has photographed the entire test subject as an optical image that is external information.

Next, in order to allow discrimination between the image determined to have necessity of the post-processes and the image determined to have no necessity of the post-processes in Step S11, the controller 111 performs display control on the test screen 113a (Step S12), and finishes this processing.

Specifically, the controller 111 assigns an identification mark or the like to the image determined to have necessity of the post-processes.

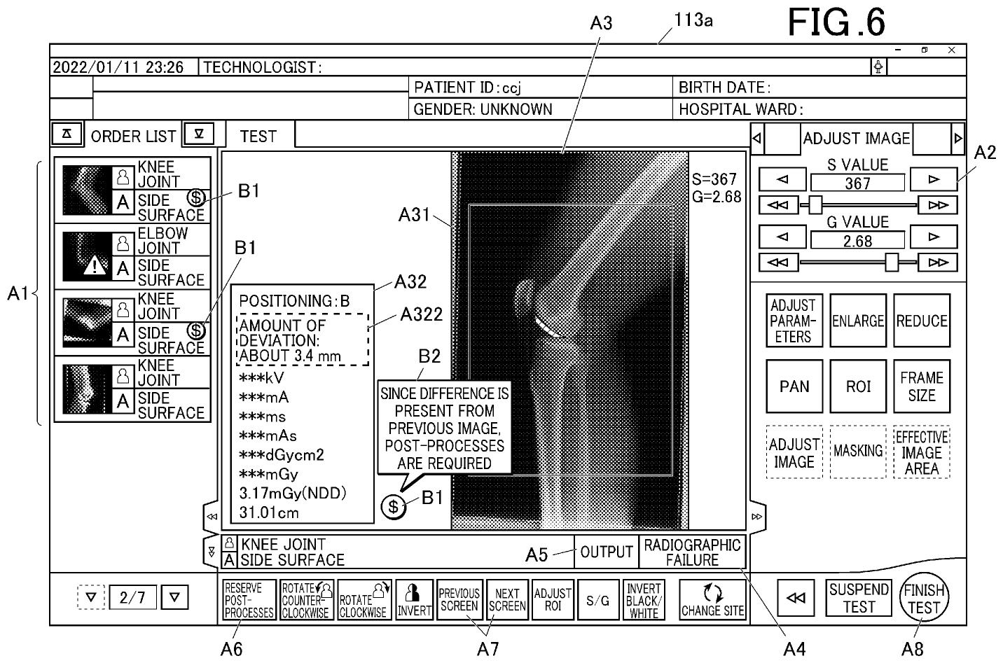

For example, as shown in FIG. 6, the controller 111 assigns a post-process reservation mark B1, as the identification mark, to each image determined to have necessity of the post-processes. Note that the post-process reservation marks of B1 are shown as a notation, e.g., “$” in FIG. 6, and the post-process reservation mark is shown similarly in the subsequent drawings.

Note that the controller 111 may assign an identification mark or the like indicating that the post-processes are not required, to the image determined to have no necessity of the post-processes.

The controller 111 may assign characters or symbols, instead of the identification mark, to the image.

Alternatively or additionally, the controller 111 may display the identification mark, or characters or symbols for identification, on any of the radiographing selection button A1 and a thumbnail or a dialog display in the radiographing selection button A1, instead of the inside of the image. In a case where one time of radiographing corresponding to the radiographing selection button A1 is accompanied by a plurality of images, the controller 111 assigns the identification mark, or characters or symbols for identification, to the radiographing selection button A1 upon determination that the post-processes are required to at least one image among the plurality of images.

Alternatively, by changing the color or shape of the radiographing selection button A1, the image determined to have necessity of the post-processes and the image determined to have no necessity of the post-processes may be discriminable from each other.

Note that in the example shown in FIG. 6, a plurality of test orders on one patient having a patient ID “ccj” are indicated, and each image that requires the post-processes among the images taken by the test orders is assigned the identification mark. There is no limitation to this. Alternatively, on a test list screen (not shown) where each test order is displayed in one line, each test order accompanied by any image requiring the post-processes may be assigned the identification mark or the like.

As shown in FIG. 6, in Step S11, the controller 111 may display a reason B2 of determining whether or not the post-processes are required to be applied to the image taken this time, in the test screen 113a, in an overlaid manner, as a comment or the like.

The controller 111 may display only the image determined to have necessity of the post-processes, in the image display area A3.

For example, when the controller 111 switches the image displayed in the image display area A3 by the switching button A7, the controller 111 regards each image determined to have no necessity of the post-processes as an image that is not to be switched by the switching button A7 (unswitchable). When predetermined radiographing is selected from the test order list by the radiographing selection button A1, the controller 111 may allow display in the image display area A3 even if the image corresponding to the radiographing is unswitchable.

A case is described where radiography this time is dynamic radiography, and the controller 111 determines whether or not to have necessity of the post-processes in units of frame images, and displays, on the display 113, only each frame image determined to have necessity of the post-processes. This is, for example, a case where a range of images to be examined among all the frame images in one time of radiographing (a range to be provided to the clinician etc.) (e.g., a case of allowing limitation to one cycle of respiration etc.).

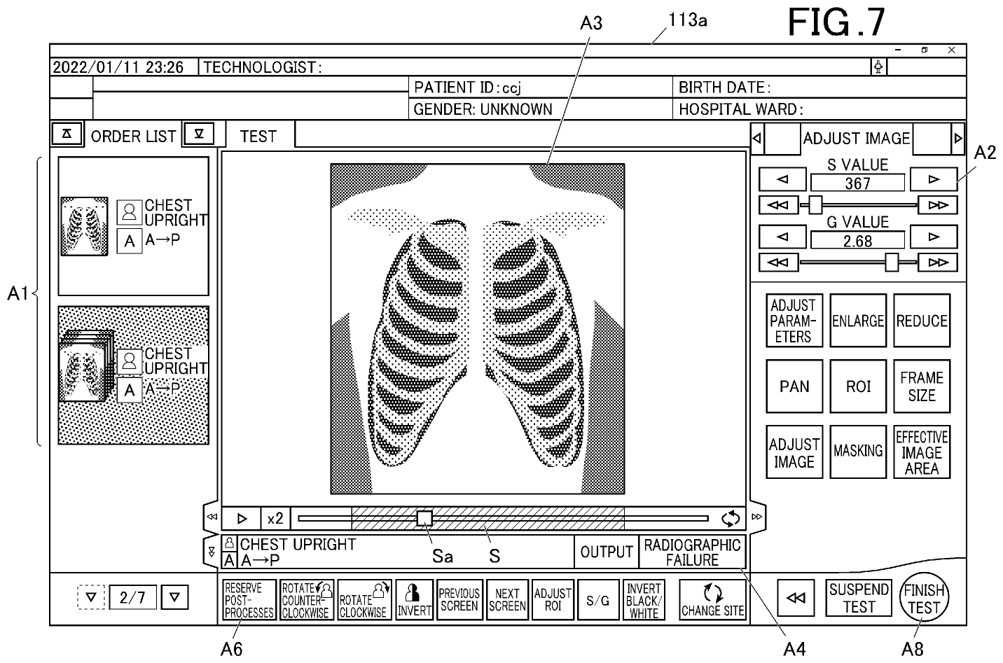

In this case, as shown in FIG. 7, a seek bar (reproduction bar) S is provided at the bottom of the test screen 113a. A slider Sa displayed on the seek bar S indicates the position of the frame image currently displayed in the image display area A3 in the entire dynamic image.

The controller 111 displays a range of the frame images determined to have necessity of the post-processes, and a range of the frame images determined to have no necessity of the post-processes, in different colors, on the seek bar S. In the example shown in FIG. 7, the range of the frame images determined to have necessity of the post-processes is displayed with hatching, and the range of the frame images determined to have no necessity of the post-processes is displayed in white. The slider Sa can move only the range displayed with hatching (the range of the frame images determined to have necessity of the post-processes).

In a case where radiography this time is a dynamic radiography, and the frame image displayed in the image display area A3 is switched by the switching button A7, the controller 111 performs switching, within a limited range of the frame images determined to have necessity of the post-processes. That is, the controller 111 regards the frame images determined to have no necessity of the post-processes as images out of the switchable target by the switching button A7 (unswitchable).

A case is described where radiography this time is dynamic radiography, and the controller 111 determines whether or not to have the necessity of the post-processes in units of frame images, and determines that all the frame images in one time of radiography are targets of the post-processes as a result of the determination. This is, for example, a case where one time of radiography does not reach one cycle of respiration, or a case where the frame images determined to have necessity of the post-processes require the time series of previous and subsequent frame images, or are required to be subjected to a difference process. Specifically, this is a case where the gradation process is required for the frame image with the maximum inspiration. When viewed as a moving image, the gradation is required to conform also to the previous and subsequent frame images. Accordingly, the post-processes are required also to the frame images other than the frame images determined to have necessity of the post-processes. As a result, the post-processes are sometimes required for all the frame images. In this case, the controller 111 can display all the frame images on the display 113.

Also in the case described above, if the test purpose and analysis purpose are preliminarily confirmed by the test order information or the like, the controller 111 determines whether or not the range of the frame images requiring the post-processes can be identified in conformity with the test purpose and analysis purpose. If the range can be identified, only the frame images requiring the post-processes may be displayed on the display 113 as shown in FIG. 7.

The controller 111 performs display control on the test screen 113a so that processes determined not to be required to the post-processes in Step S11 are inexecutable.

For example, as shown in FIG. 6, the controller 111 grays out some of buttons (represented by broken lines in FIG. 6) in the setting area A2, and prevents the buttons from being selected. Note that the controller 111 may hide buttons for selecting the processes determined not to be required for the post-processes, on the test screen 113a. Note that the controller 111 may only display buttons for selecting the processes determined to be required for the post-processes, on the test screen 113a.

Note that in Step S11, the controller 111 may assign the degree of necessity (priority) to each image determined to have necessity of the post-processes. The priority is, for example, “the post-processes are required”, “the post-processes are recommended”, “confirmation is recommended just in case” or the like, and may be input by the user through the operation receiver 114, or be set by the controller 111.

In Step S12, the controller 111 performs the display control in accordance with the priority set in Step S11. Specifically, the controller 111 makes the priority identifiable by assigning the image a mark or characters, or changing the color of the image in accordance with the priority. The characters are, for example, “confirmation is required”, “just in case” or the like.

In Step S12, the controller 111 may perform display control in accordance with the processes determined to be required among the post-processes in Step S11. For example, the processes may be gradation adjustment, output destination review, readjustment of the masking range, etc.

Specifically, the controller 111 makes the difference between the processes identifiable by assigning a mark or characters to the image, or changing the color of the image in accordance with each process determined to be required.

The controller 111 may display, in the test screen 113a, a comment or the like in accordance with each process determined to be required. The comment may be input by the user through the operation receiver 114, or be set by the controller 111.

Returning to FIG. 3, after the post-process determination process is finished, the radiographer releases the test subject from the state of being positioned.

Next, the controller 111 accepts the user's execution of the post-processes to the image determined in Step S5 to have necessity of the post-processes (Step S6), and transfers the processing to Step S1, thus allowing transition to the next radiographing. That is, the controller 111 accepts execution of the predetermined processes (post-processes) by the user to the radiograph identified by the identifier. Here, the user may be a technologist, a radiologist, a diagnostician or the like, other than the radiographer. By separating a user performing execution of radiography, radiographic failure determination, and determination of the necessity of the post-processes, from a user executing the post-processes, the plurality of test post-processes can be comprehensively executed in a terminal apparatus used by the user executing the post-processes and communicable to the radiography control device 11. A user performing execution of radiography and radiographic failure determination, a user performing determination of the necessity of the post-processes, and a user executing the post-processes may be different from each other.

In Step S6, the controller 111 stores, in the storage 112, a result of execution (presence or absence of execution) of the post-processes to the image.

Note that in Step S6, the controller 111 may perform the post-processes by itself to the image determined in Step S5 to have necessity of the post-processes. That is, the controller 111 executes the predetermined processes (post-processes) to the radiograph identified by the identifier.

An example is described where the controller 111 executes the post-processes by itself to the image determined in Step S5 to have necessity of the post-processes.

For example, the controller 111 executes the fixed processes (the gradation process, trimming, etc.) applied to the image taken last time by radiographing the same radiography site as the radiography site radiographed this time, to the image determined to have necessity of the post-processes.

The controller 111 assigns posture information on the test subject determined based on the optical image, to the image determined to have necessity of the post-processes, in a form of an annotation.

If the controller 111 determines that the location of the patient is in an operating room based on the patient information, the controller 111 applies the gauze emphasizing process and the like to the image determined to have necessity of the post-processes, and generates a gauze-emphasizing-processed image.

In a case where the location of the patient is an ICU (Intensive Care Unit) or an NICU (Neonatal Intensive Care Unit), it is required to be pulled out early. Accordingly, only a limited time period can be possibly taken for executing the post-processes after radiography. Consequently, when the controller 111 determines that the location of the patient is an ICU or an NICU based on the patient information, the controller 111 applies post-processes having a short processing time period, or post-processes having relatively low processing loads, to the image determined to have necessity of the post-processes. For example, if the radiography this time is for a still image, the processing load of the post-processes on the still image is relatively low, and the controller 111 executes the post-processes to the image determined to have necessity of the post-processes accordingly. On the other hand, if the radiography this time is dynamic radiography, a dynamic image includes a plurality of frame images, and the post-processes to the dynamic image have higher processing load than the post-processes to the still image do. Accordingly, the controller 111 displays the image taken this time as an original image itself on the display 113.

The controller 111 may preset the acceptable range of the processing time period of the post-processes and the types of post-processes executed by this controller to the image determined to have necessity of the post-processes, in accordance with the facility and environment.

In Step S6, when the controller 111 executes, by itself, the post-processes to the image determined in Step S5 to have necessity of the post-processes, and the post-processes are not applied by the user, the controller 111 may displays, on the test screen 113a, a notification dialog for notification that the post-processes have been automatically executed, but the post-processes by the user have not been executed, when the test finish button A8 is pressed.

In Step S6, if the test finish button A8 is pressed with the post-processes by the controller 111 or the user having not been applied to the image determined in Step S5 to have the necessity of the post-processes, the controller 111 may display, on the test screen 113a, a notification dialog for notification that the post-processes have not been executed.

(Display Process after Post-Process Execution)

Next, a display process after post-process execution for allowing the user to control display of the image to which the user has applied the post-processes in Step S6 of the aforementioned radiographing process, and the image to which the user has not applied the post-processes, is described.

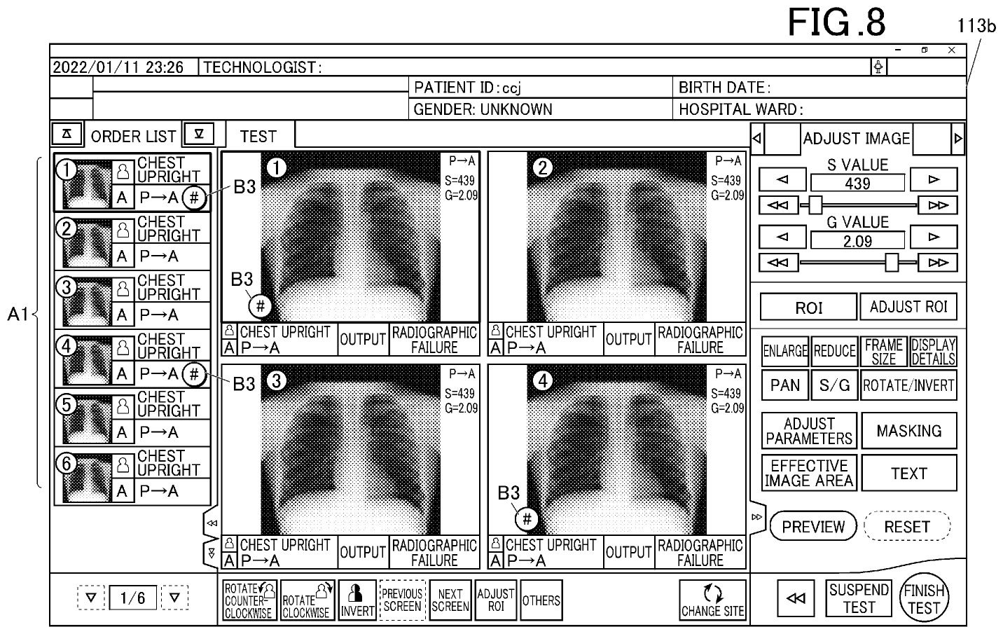

FIG. 8 shows an example of a test screen 113b after execution of the post-processes in Step S6 described above.

In the example shown in FIG. 8, numbers displayed on the respective radiography selection buttons A1 correspond respectively to numbers displayed on images displayed in the image display area A3.

In the display process after post-process execution, the controller 111 performs display control on the test screen 113b so that the images to which the user has applied the post-processes, and the images to which the user has not applied the post-processes can be discriminated from each other.

Specifically, the controller 111 assigns an identification mark or the like to each image to which the user has applied the post-processes.

For example, as shown in FIG. 8, the controller 111 assigns a processed mark B3 serving as an identification mark, to the radiography selection button A1 corresponding to each image to which the user has applied the post-processes, and each post-processed image Note that the processed marks of B3 are shown as a notation, e.g., “#” in FIG. 8, and the processed mark is shown similarly in the subsequent drawings.

Note that the controller 111 may assign an identification mark or the like indicating that the post-processes have not been applied, to the radiography selection button A1 corresponding to each image to which the user has not applied the post-processes, and each image not subjected to the post-processes.

The controller 111 may assign characters or symbols, instead of the identification mark, to each radiography selection button A1 and each image.

The controller 111 may display the identification mark, or the characters or symbols for identification, not only on the radiography selection buttons A1 and the images but also on any of the thumbnails or dialog displays in the radiography selection buttons A1. In a case where one time of radiographing corresponding to the radiographing selection button A1 is accompanied by a plurality of images, when the user applies the post-processes to at least one of the images, the controller 111 assigns the identification mark, or characters or symbols for identification, to the radiographing selection button A1.

Alternatively, by changing the color or shape of the radiographing selection button A1, the image to which the user has applied the post-processes, and the image to which the user has not applied the post-processes may be discriminable from each other.

Note that ON/OFF of identification display in the display process after post-process execution, the method for displaying the identification, the screen configuration of the test screen 113b and the like may be set by an input operation by the user, or preset for each of facilities, each of technologists, each of test types, or each of departments.

The display process after post-process execution allows the user to easily discriminate the image to which the user has applied the post-processes from the image to which the user has not applied the post-processes. Accordingly, the user can perform confirmation in view of whether or not the post-processes are not required to the image not subjected to the post-processes, in actuality. Furthermore, the user can perform confirmation in view of whether or not the post-processes applied to the post-processed image were appropriate.

In the display process after post-process execution described above, the controller 111 may display the images to which the user has applied the post-processes, and the unprocessed images (original image) of these images side-by-side on the test screen 113b.

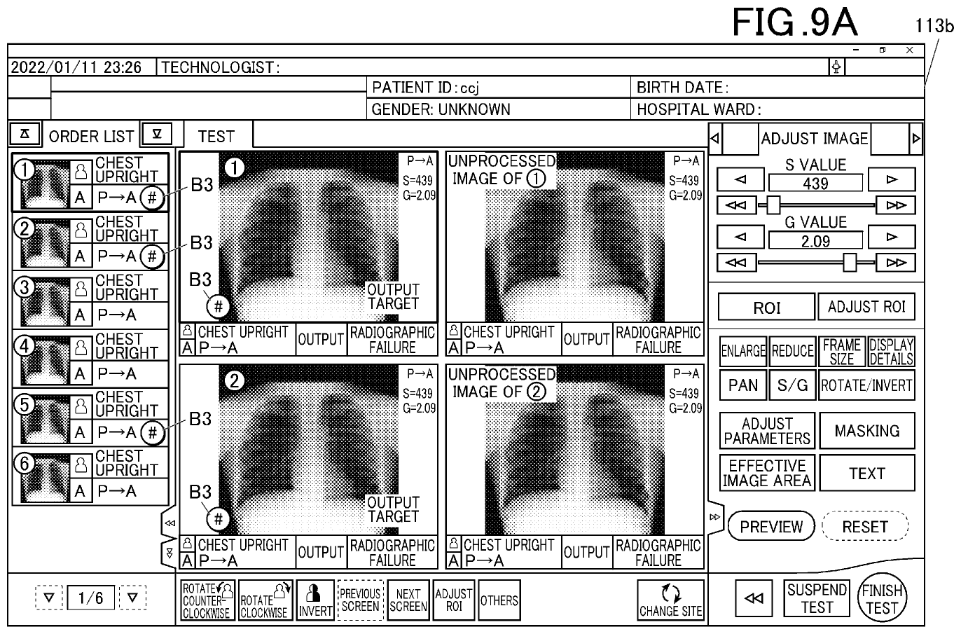

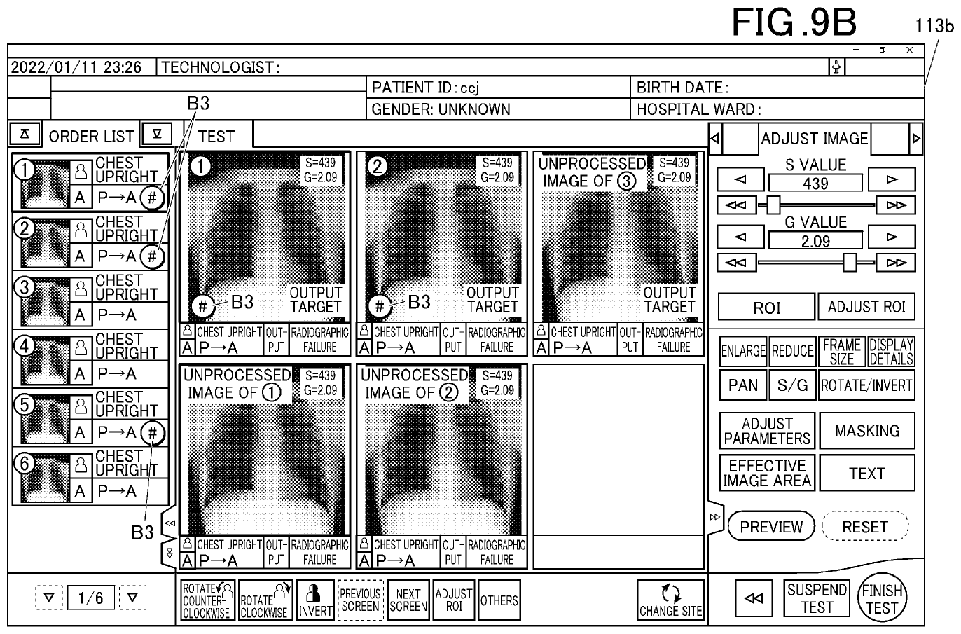

FIGS. 9A and 9B show examples of the test screen 113b where the images to which the user has applied the post-processes, and their original images are displayed side-by-side in the image display area A3.

In the example shown in FIG. 9A, the controller 111 displays the images to which the user has applied the post-processes, and their original images side-by-side in the image display area A3. The controller 111 displays the output-target images on the left side of the image display area A3.

In the example shown in FIG. 9B, the controller 111 displays the images to which the user has applied the post-processes, and their original images in tandem in the image display area A3. For an image not subjected to the post-processes, such as the third image shown in FIG. 9B, the controller 111 displays only an unprocessed image. The controller 111 also displays the output-target images at an upper part of the image display area A3.

With reference to the display described above, the user compares each image to which the user has applied the post-processes with its unprocessed image (original image), and can perform confirmation in view of whether or not the applied post-processes were appropriate.

Note that the controller 111 may display only the images to which the user has applied the post-processes, in the image display area A3.

The controller 111 may display each image to which the user has applied the post-processes, and its original image side-by-side in another dialog or another screen instead of or in addition to the image display area A3.

In the display process after post-process execution described above, the controller 111 may display, on the test screen 113b, the images to which the user has applied the post-processes, and images temporarily generated in radiographing of the images and in the post-processes. The temporarily generated images may be, for example, a long-time image before combination, a radiographic failure image, an image subjected to predetermined frequency emphasizing processes (e.g., the catheter distal end emphasizing process, and the gauze-emphasizing-processed image), etc.

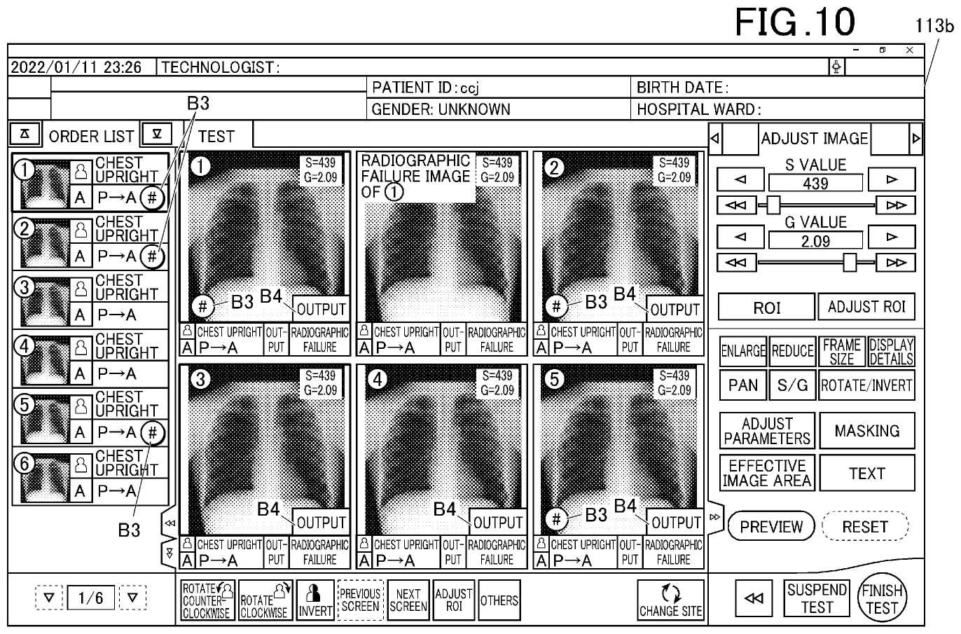

FIG. 10 shows an example of the test screen 113b where the image to which the user has applied the post-processes, and a temporarily generated image (radiographic failure image) are displayed in the image display area A3.

As shown in FIG. 10, the controller 111 assigns output-target images identification information B4 that is a mark, characters or the like for discriminating the output-target images from non-output-target images Note that the identification information B4 may be a frame of the corresponding image, or display in a different color.

In a case where it is only intended to discriminate the output-target image from the non-output-target image, the processed mark B3 may be omitted. However, it is preferable to assign the processed mark B3 to each temporarily generated image because the assignment allows recognition of whether the image is the image generated by the user's post-processes or the image generated by automatically applying post-processes (by the controller 111).

The display described above allows the user to verify whether or not the temporarily generated image is an output target.

Note that the controller 111 may display the image to which the user has applied the post-processes, and the temporarily generated image, in another dialog or another screen instead of or in addition to the image display area A3.

If the numbers of images to which the user has applied the post-processes and temporarily generated images are large, and all of the images to which the user has applied the post-processes and the temporarily generated images cannot be completely displayed in the test screen 113b, the controller 111 may provide a slider bar on the test screen 113b, and switch the displayed image. The controller 111 may increase the number of images displayable on the test screen 113b.

In the display process after post-process execution described above, the controller 111 may display each image to which the user has applied the post-processes, and the previous image of the image concerned, on the test screen 113b. The previous image of the image concerned is the previous image taken by radiographing the same radiography site as the radiography site of the image to which the user has applied the post-processes.

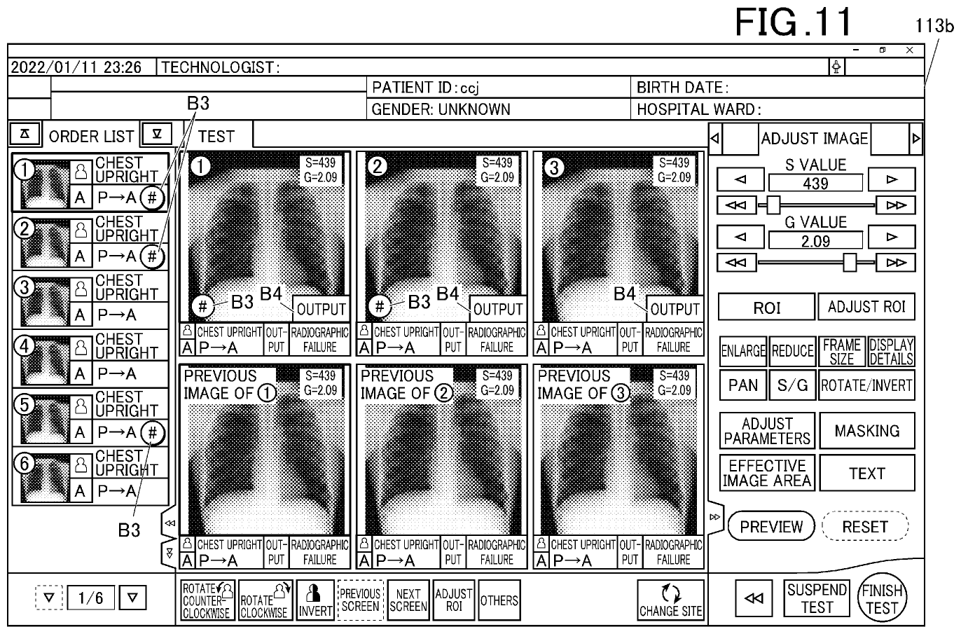

FIG. 11 shows an example of the test screen 113b where the images to which the user has applied the post-processes, and the previous images of the images concerned are displayed in tandem in the image display area A3. As with the third image shown in FIG. 11, the controller 111 may display each image to which the user has not applied the post-processes, and the previous image of the image concerned in tandem. The controller 111 displays the output-target images at an upper part of the image display area A3.

With respect to the display described above, once more before each image is output, the user compares the image to which the user has applied the post-processes with the previous image of the image concerned, and can perform confirmation in view of whether or not the applied post-processes were appropriate.

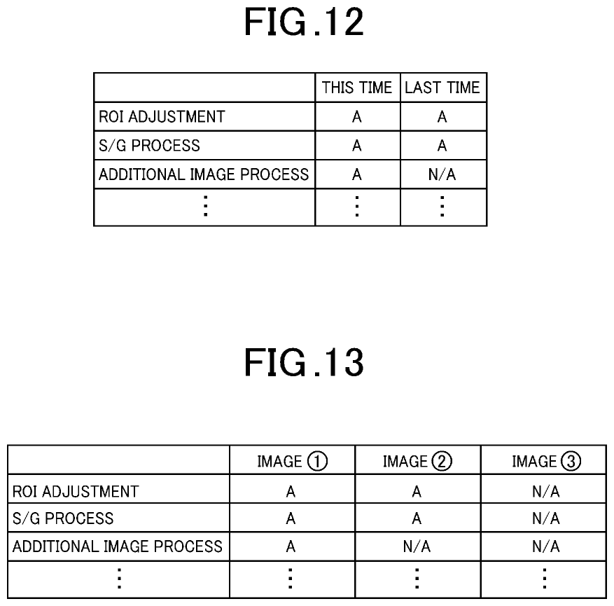

Note that the controller 111 may display, on the test screen 113b, information on presence or absence of application of the post-processes to the previous image corresponding to the image to which the user has applied the post-processes, and the content of the applied post-processes (post-process types). In this case, the controller 111 may display, on the test screen 113b, a table of comparing the post-processes applied to the image taken this time with the post-processes applied to the image taken last time, as shown in FIG. 12. In the example shown in FIG. 12, A and N/A respectively indicate presence and absence of the processes.

In the display process after post-process execution described above, the controller 111 may display the content of the post-processes applied by the user (post-process types), on the test screen 113b.

FIG. 13 shows a table indicating the content of the post-processes applied by the user to first to third output-target images Note that in the table indicating the content of the post-processes, values changed by the processes may be displayed instead of indication of presence and absence of execution of the processes by A and N/A.

When a predetermined image is selected by the user's input operation on the test screen 113b, the controller 111 may display, in the setting area A2, the content of the post-processes applied to the selected image.

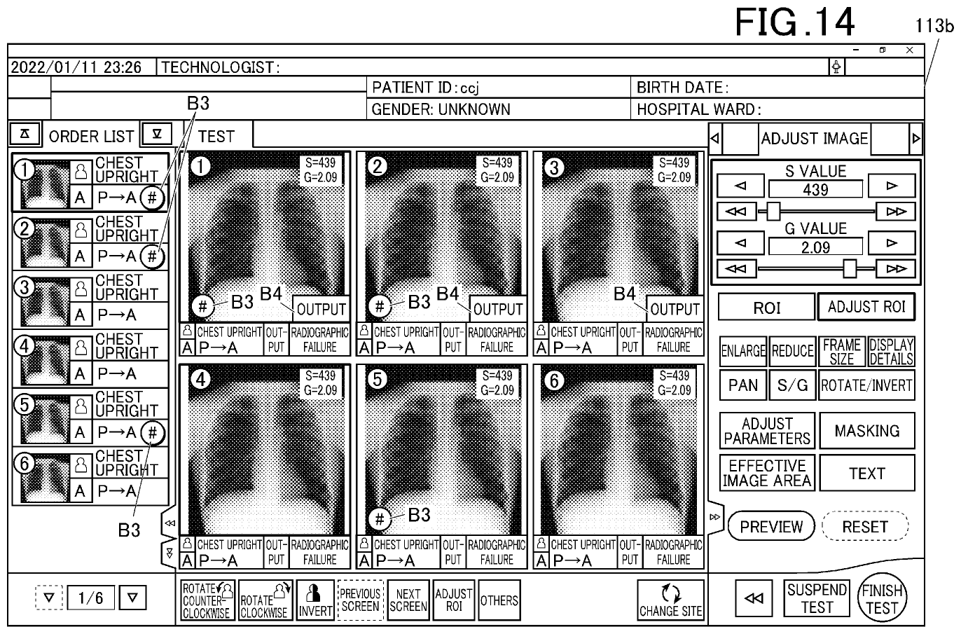

FIG. 14 shows an example of the test screen 113b where the content of the post-processes is displayed in the setting area A2.

In the example shown in FIG. 14, the controller 111 assigns a black frame to each button for selecting a process executed in the post-processes, in the setting area A2. Note that the controller 111 may change the color of each button for selecting a process executed in the post-processes.

The display described above allows the user to comprehensively confirm the content of the post-processes applied to the output target image before output of the image.

As described above, by executing the display process after post-process execution, the user can confirm the execution result of the post-processes by the user more effectively while maintaining the viewability of the plurality of images. Furthermore, the user can effectively execute final confirmation of whether the output-target image to the PACS or the like is an appropriate image in conformity with the test order.

### Second Embodiment

Next, an X-ray radiographic system 1 according to a second embodiment is described. Hereinafter, points different from those of the first embodiment are mainly described.

In Step S2 of the radiographing process in the X-ray radiographic system 1 according to the embodiment, the controller 111 obtains the radiographing condition included in the test order selected by the radiographer, from the storage 112, transmits the radiographing condition to the X-ray generating control device 21 and the FPD 12, and configures the setting. That is, the controller 111 obtains the radiographing condition for a radiograph. Here, the controller 111 functions as a second obtainer.

Next, in Step S3 of the radiographing process, the controller 111 performs radiography, and obtains a taken image from the FPD 12. That is, the controller 111 obtains a radiograph. Here, the controller 111 functions as a first obtainer. The radiograph may be a plurality of frame images taken by one time of dynamic radiographing.

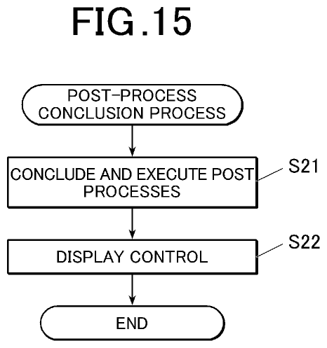

Next, in Step S5 of the radiographing process, the controller 111 executes a post-process conclusion process shown in FIG. 15 instead of the post-process determination process.

### (Post-Process Conclusion Process)

In the post-process conclusion process, the controller 111 concludes the types of post-processes to be applied to the image (taken this time) taken in Step S3, and the processing condition, based on the radiographing condition, and applies the concluded post-processes to the image taken this time (Step S21). If the radiography taken this time is dynamic radiography, the controller 111 executes the process in Step S21 in units of frame images.

That is, the controller 111 automatically concludes the types of predetermined processes (post-processes) to be executed by the processor (controller 111), and the processing condition based on the radiographing condition obtained by the second obtainer. Here, the controller 111 functions as a concluder.

The controller 111 then executes predetermined processes (post-processes) determined by the concluder. Here, the controller 111 functions as a processor.

Note that in Step S4 of the radiographing process, the post-process conclusion process may be executed in parallel with the radiographic failure determination at the same time.

The post-process type is any of ROI (Region Of Interest) adjustment, effective image area setting, S/G value adjustment, rotation/inversion/rotate by any angle, image processing (E process/F process/H process/scattered radiation correction/image processing condition changing, etc.), emphasizing processes (a catheter distal end emphasizing process, a gauze emphasizing process, and/or other emphasizing processes, etc., as frequency emphasizing processes), and a grid removal process, masking, trimming, marker/stump/overlay, and output setting.

The processing condition of the post-processes described above is parameters in each process, and is a frequency band or the like emphasized in a frequency emphasizing process, for example.

In Step S21, the controller 111 concludes the post-process types and the processing condition, based on the post-processes applied to the image taken last time by radiographing the same radiography site as the radiography site radiographed this time, and executes the concluded post-processes to the image taken this time.

Specifically, the controller 111 concludes that the post-process types and the processing condition that are identical to the types of the post-processes executed to the image taken last time and the processing condition are applied to the image taken this time.

For example, the controller 111 executes position adjustment and image processing to the image taken this time so that the effective image area and the ROI of the image taken this time can be the same as those of the last image.

If the image taken this time satisfies a specific condition, the controller 111 concludes the post-process types and the processing condition, and executes the concluded post-processes to the image taken this time. The specific condition is a specific radiography site, a specific case, a respiratory cycle, and the like.

For example, if the radiography site of the image taken this time is a side surface of a knee joint, the controller 111 executes position adjustment to the image taken this time so that the articular surfaces of internal and external condyles of a femur can be positioned at the center of the image.

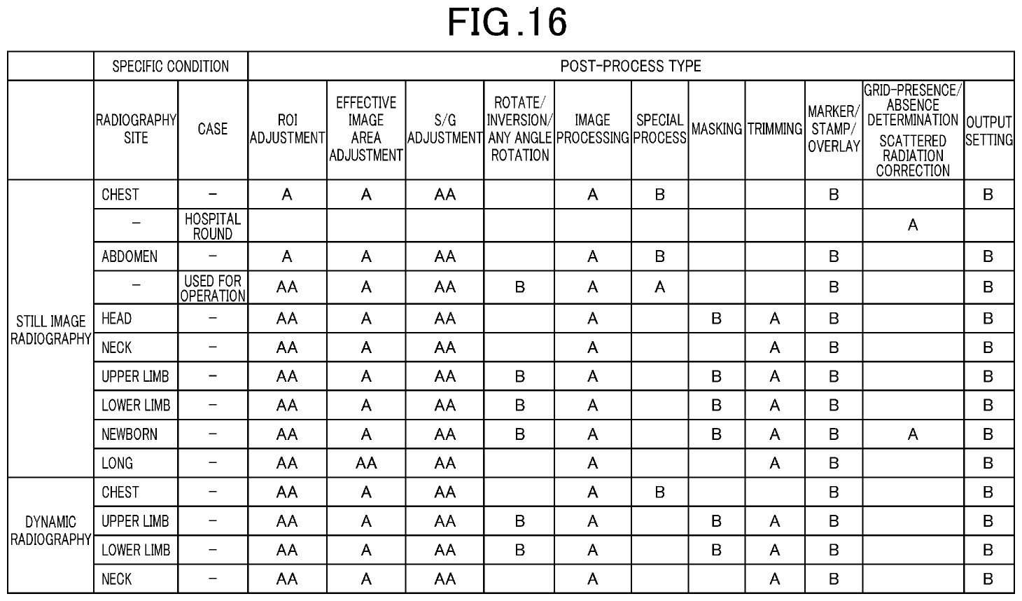

For example, FIG. 16 shows an example of types of the post-processes executed by the controller 111 with respect to each specific radiography site and to each specific case.

In the example shown in FIG. 16, the descending order of priorities of the post-process execution is indicated by AA, A, and B. Blank fields indicate that no process is executed.

The controller 111 concludes the post-process types and the processing condition, based on a reference image taken by radiographing the same radiography site as the radiography site radiographed this time, and executes the concluded post-processes to the image taken this time.

The reference image is an appropriate image for a clinician and a radiologist (an image intended to be viewed, and a viewable image).

Specifically, the controller 111 extracts the difference between the image taken this time and the reference image, and executes the post-processes to the image taken this time so as to eliminate the difference. The processes are executed by machine learning with the reference image being adopted as training data.

The controller 111 concludes the post-process types and the processing condition, based on external information, and executes the concluded post-processes to the image taken this time.

For example, a case is described where the X-ray radiographic system 1 includes an optical camera (not shown) that takes an optical image in a range including at least a part of an area irradiated with X-rays by the X-ray tube device 25. In this case, the controller 111 determines the radiographing direction of the image taken this time, based on the optical image that is external information, and applies a process of assigning, as an annotation, the determined radiographing direction to the image taken this time. In particular, if the radiography site this time is a head or a neck, the controller 111 may apply the process of assigning the radiographing direction as an annotation.

The controller 111 concludes the post-process types and the processing condition, based on statistical information, and executes the concluded post-processes to the image taken this time. The statistical information is, for example, statistical information on radiographic failure reasons, and QA (quality assurance) feedback reasons for previously taken images.

Specifically, if the QA feedback reason of the image taken last time by radiographing the same radiography site as the radiography site radiographed this time is disagreement in the trimming size, the controller 111 adjusts the trimming size for output of the image taken this time so as to make the trimming sizes coincide.

If the QA feedback reason for the previously taken image is that neither a catheter-distal-end-emphasizing-processed image nor a gauze-emphasizing-processed image are applied when the image is output, the controller 111 generates a catheter-distal-end-emphasizing-processed image, and a gauze-emphasizing-processed image of the image taken this time, assigns the generated catheter-distal-end-emphasizing-processed image and gauze-emphasizing-processed image to the image taken this time, and outputs the images.

If the X-ray radiographic apparatus 10 and the X-ray generation apparatus 20 do not cooperate with each other, the controller 111 applies the processes similar to the image processing applied to the previously taken images, to the image taken this time to which image processing based on the irradiation condition and grid information during the radiography has not been applied yet. For example, if the grid removal process has been applied to the previously taken images at a statistically high rate, the controller 111 applies the grid removal process to the image taken this time. Instead of or adding to the application of the grid removal process to the image taken this time, the controller 111 may display a dialog for verifying whether or not to apply the grid removal process to the image taken this time when outputting the image taken this time.

When the test finish button A8 is pressed by the user, the controller 111 may display whether or not to adjust so that the trimming size for output of the image taken this time coincides with the statistically frequent trimming size among the trimming sizes of previously taken images, in a dialog display in a test screen 113c.

The controller 111 may conclude the post-process types and the processing condition, based on statistical information integrally collected by a statistical aggregating function of the X-ray generation console 22, and execute the concluded post-processes to the image taken this time.

For example, the controller 111 may determine whether or not to automatically apply the concluded post-processes, on a user-by-user basis, based on the number of radiographic failures about each user, which is statistical information.

For example, the controller 111 may determine whether or not to execute the post-processes causing a radiographic failure reason, to the image taken this time, based on the radiographic failure reason as the statistical information.

For example, the controller 111 may perform adjustment so that the S value, G value, EI value, TI value and the like of the image taken this time can be the same as the S value, G value, EI value, TI value and the like that are statistical information on the previous images taken by radiographing the same radiography site as the radiography site radiographed this time.

For example, the controller 111 may conclude the post-process types and the processing condition, based on the use frequencies of the FPD 12, the X-ray generation apparatus 20, the grid and the like, which are statistical information, and determine whether or not to execute the concluded post-processes to the image taken this time. Specifically, the controller 111 concludes the post-process types and the processing condition for correcting the adverse effects due to deterioration of the FPD 12, the X-ray generation apparatus 20, the grid and the like.

The statistical information described above may be statistical information based on the user's input operation, or statistical information integrally collected through an operation log and the like. The data aggregating method about the statistical information may be any manner. As to the statistical information, selection of items used to conclude the post-process types and the processing condition, and a determination policy of whether or not to execute the concluded post-processes on an item-by-item basis may be designatable through preliminary setting with respect to each facility, each console apparatus, each user, each radiographing room and the like. The priority order of the items of the statistical information that are used to conclude the post-process types and the processing condition may be designatable by the facility, apparatus, and department concerned.

The controller 111 may conclude the post-process types and the processing condition by the predetermined method described above, and execute the determined post-processes to the image taken this time, based on the user's input operation.

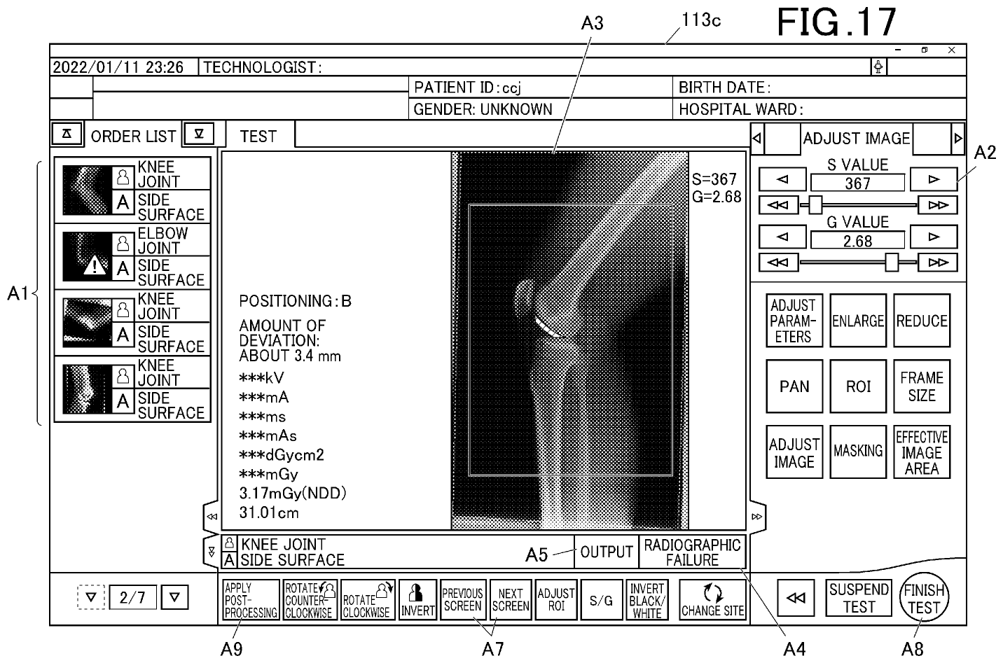

FIG. 17 shows an example of the test screen 113c displayed on the display 113.

The test screen 113c is provided with the radiography selection buttons A1, the setting area A2, the image display area A3, the radiographic failure button A4, the output button A5, the switching buttons A7, the test finish button A8, a post-process application button A9 for an instruction for executing the post-processes, and the like.

Specifically, the controller 111 accepts an instruction by the user for applying the concluded post-processes to the image taken this time, through the post-process application button A9. If the user determines to apply the post-process to the image taken this time, they press the post-process application button A9. When the post-process application button A9 is pressed, the controller 111 executes the concluded post-processes to the image taken this time. The concluded post-processes executed by the controller 111 when the post-process application button A9 is pressed may be multiple types of post-processes, or multiple times of post-processes. It may be assumed to be preset, by the user, whether or not to apply the concluded post-processes to the image taken this time.

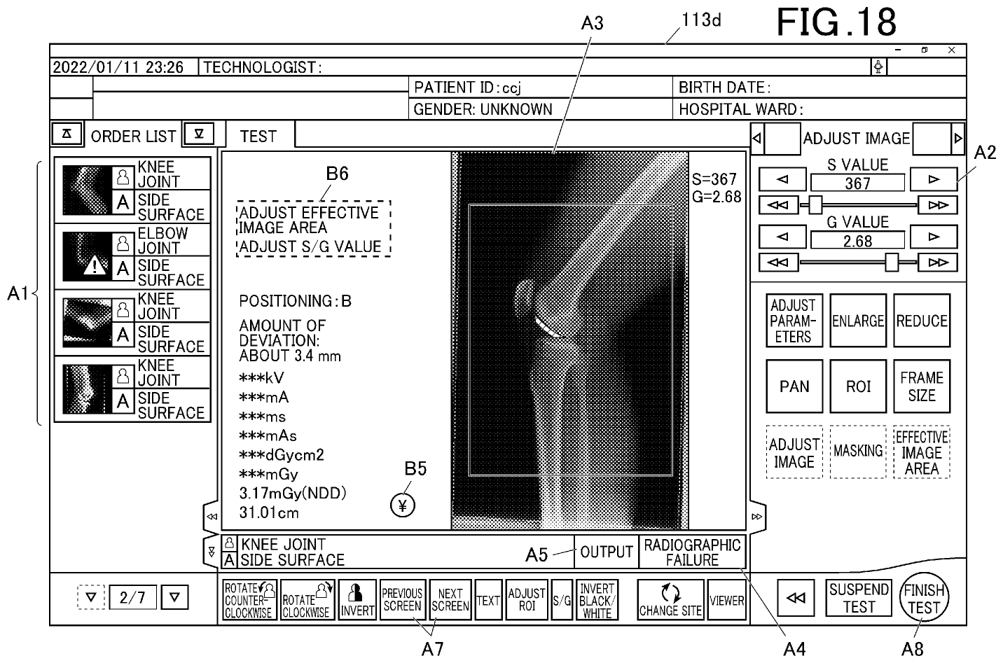

Next, in order to allow discrimination between the image to which the post-processes have been applied in Step S21, and the image to which the post-processes have not been applied, the controller 111 performs display control on a test screen 113d (see FIG. 18) (Step S22), and finishes this processing. That is, the controller 111 displays, on the display 113, the radiograph for which the post-processes (predetermined processes) have been executed by the processor. Here, the controller 111 functions as a display controller.

Specifically, the controller 111 assigns an identification mark or the like to each image to which the post-processes have been applied.

For example, as shown in FIG. 18, the controller 111 assigns a post-process execution mark B5, as an identification mark, to the image to which the post-processes have been applied. Note that the post-process execution mark of B5 is shown as a notation, e.g., Yen mark in FIG. 18.

Note that the controller 111 may assign an identification mark or the like indicating that the post-processes have not been executed, to the image to which the post-processes have not been applied.

The controller 111 may assign characters or symbols, instead of the identification mark, to the image.

The controller 111 may also display the identification mark, or characters or symbols for identification, on any of the radiographing selection button A1 and a thumbnail or a dialog display in the radiographing selection button A1, instead of or adding to the inside of the image. In a case where one time of radiographing corresponding to the radiographing selection button A1 includes a plurality of images, the controller 111 assigns the identification mark, or characters or symbols for identification, to the radiographing selection button A1 when the post-processes are applied to at least one of the plurality of images. Alternatively, by changing the color or shape of the radiographing selection button A1, the image to which the post-processes have been applied, and the image to which the post-processes have not been applied may be discriminable from each other.

As shown in FIG. 18, in Step S21, the controller 111 may display, in the test screen 113d, the content of the post-processes (post-process types) B6 having applied to the image taken this time.

In Step S21, the controller 111 performs display control for the test screen 113d so that the user cannot additionally execute the post-processes having been applied to the image taken this time.

For example, as shown in FIG. 18, the controller 111 grays out some of buttons (represented by broken lines in FIG. 18) in the setting area A2, and prevents the buttons from being selected. Note that the controller 111 may hide buttons for selecting the post-processes having been applied to the image taken this time, on the test screen 113d.

For example, when the image taken this time is used for a surgical operation, the controller 111 displays the catheter-distal-end-emphasizing-processed image, and the gauze-emphasizing-processed image of the image taken this time, on the test screen 113d.

For example, the controller 111 displays the catheter-distal-end-emphasizing-processed image, the gauze-emphasizing-processed image, and their original image, on the test screen 113d in an arranged manner.

The controller 111 displays, on the test screen 113d, images obtained by changing the emphasizing levels of the catheter-distal-end-emphasizing-processed image and the gauze-emphasizing-processed image in an arranged manner.

For example, if the image taken this time is used for a surgical operation, the controller 111 displays the image taken before the surgical operation, on the test screen 113d.

As described above, the radiography control device 11 (control device) includes: an obtainer (controller 111) that obtains a plurality of radiographs satisfying a predetermined condition; and an identifier (controller 111) that identifies a radiograph to be subjected to the predetermined processes (post-processes), among the plurality of radiographs obtained by the obtainer.

Consequently, since the radiograph required to be subjected to the post-processes can be automatically identified, the task of executing the predetermined processes (post-processes) to the radiograph can be effectively executed.

In the radiography control device 11, the predetermined condition is any of a radiographic target being an identical test subject, an identical radiography date, an identical radiography site, and an identical user.

Consequently, even if a large number of radiographing operations are performed a day, since the radiograph required to be subjected to the post-processes can be automatically identified, the task of executing the predetermined processes (post-processes) for the radiograph can be effectively executed.

In the radiography control device 11, the identifier identifies the radiograph to be subjected to the predetermined processes (post-processes), based on information on images previously taken by radiographing the identical radiography site.

Consequently, a radiograph required to be subjected to the post-processes can be more appropriately identified based on the images previously taken by radiographing the identical radiography site.

In the radiography control device 11, the identifier identifies an image corresponding to at least any of a predetermined radiography site, predetermined usage, and a respiratory cycle, as the radiograph to be subjected to the predetermined processes (post-processes).

Consequently, a radiograph required to be subjected to the post-processes can be more appropriately identified based on the specific condition (the predetermined radiography site, predetermined usage, and respiratory cycle).

In the radiography control device 11, the identifier identifies the radiograph to be subjected to the predetermined processes (post-processes), based on an instruction by the user.

Consequently, the user can identify the radiograph determined to be subjected to the post-processes, as the radiograph to be subjected to the post-processes.

In the radiography control device 11, the identifier identifies the radiograph to be subjected to the predetermined processes (post-processes), based on external information.

Consequently, a radiograph required to be subjected to the post-process can be more appropriately identified based on external information.

The radiography control device 11 includes a display controller (controller 111) that presents a display that discriminates the radiograph that is identified by the identifier to be subjected to the predetermined processes (post-processes), from the radiograph that is not identified by the identifier.

Consequently, the user can easily discriminate the radiograph required to be subjected to the post-processes, from the radiograph to which the user is not required to execute the post-processes.

In the radiography control device 11, the display controller does not display the radiograph that is not identified by the identifier, during execution of the predetermined processes (post-processes).

Consequently, the post-processes can be prevented from being executed to the radiograph to which the user is not required to execute the post-processes.

The radiography control device 11 includes a processor (controller 111) that executes the predetermined processes (post-processes) to the radiograph that is identified by the identifier to be subjected to the predetermined processes (post-processes).

Consequently, the predetermined processes can be effectively executed to the plurality of radiographs determined to have the necessity of the post-processes.

The radiography control device 11 includes an acceptor (controller 111) that accepts execution of the predetermined processes (post-processes) by the user to the radiograph that is identified by the identifier to be subjected to the predetermined processes (post-processes).

Consequently, the predetermined processes can be effectively executed to the plurality of radiographs determined to have the necessity of the post-processes.

In the radiography control device 11, the plurality of radiographs include a plurality of frame images Consequently, also in dynamic radiography, the radiograph required to be subjected to the post-processes can be automatically identified. Accordingly, the task of executing the predetermined processes to the radiograph can be effectively executed.

For example, the controller 111 may store, in the storage 112, the ratio of target cases requiring the post-processes in each test, a time period from positive determination of the necessity of post-processes to execution of the post-processes, or the number of images which have not been determined to have necessity of the post-processes but were applied with the post-processes, then calculate statistical information accompanying the post-processes from information for determining the necessity of post-processes and its auxiliary information (the determination target, determination date), and a post-process execution result after the determination of the necessity of post-processes (execute or not, execution date), and display the information on the display 113 or output the information to an external apparatus. Accordingly, it can be quantitatively referred to whether or not the post-processes have effectively been performed, and the necessity of the post-processes and a notification for execution of the post-processes in accordance with each site and each condition can be shared.

For example, the X-ray radiographic system 1 may include a display terminal apparatus not shown and serving as a display, instead of or adding to the display 113. The display terminal apparatus is, for example, a mobile terminal or the like. In this case, the display terminal apparatus may display only radiographs determined to have necessity of post-processes in a limited manner, for the sake of security measures in a case of being carried outside, or for the sake of allowing effective operation in a smaller display area than that of the display 113.

The display terminal apparatus may have a function as a processor, and display the same content both on the display 113 and the display terminal apparatus. Accordingly, information required for the post-processes can be mutually shared, and for example, a sharing operation can be achieved, in which the radiography control device 11 (controller 111) determines only the necessity of the post-processes and the display terminal apparatus performs the post-processes to the radiographs having the necessity of the post-processes.

Determination of the necessity of the post-processes and conclusion of the post-process types and the processing condition are not necessarily performed in the radiography control device 11 (controller 111), and may be executed in the display terminal apparatus instead. Alternatively, the determination result of the necessity of the post-processes, and the determined post-process types and processing condition may be associated with the taken image and transmitted to another control device, and another user may execute the post-processes in the control device in an environment different from that of the X-ray radiographic system 1. Accordingly, in a case of an inexperienced technologist, and in labor shortage hours, the post-processes can be transferred to another person in charge, which facilitates load distribution.

### Third Embodiment

Next, an X-ray radiographic system 1 according to a third embodiment is described.

### (Operation of X-Ray Radiographic System)

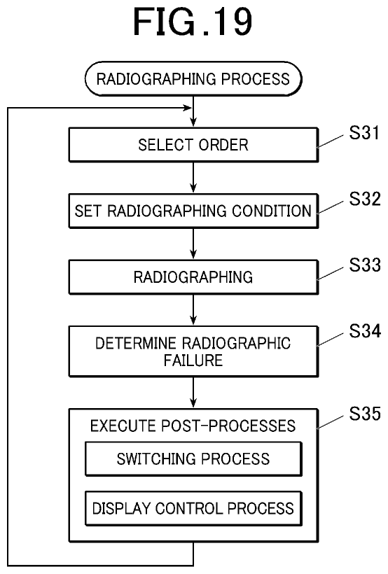

A radiographing process in the X-ray radiographic system 1 shown in FIG. 19 is described.

Here, it is assumed that test orders are preliminarily registered in the radiography control device 11. The test order may be input from an external system, such as the HIS 32 or the MS 33, or manually input by a radiographer as a user (a technologist, a radiologist, a diagnostician, etc.) through the operation receiver 114.

### (Radiographing Process)

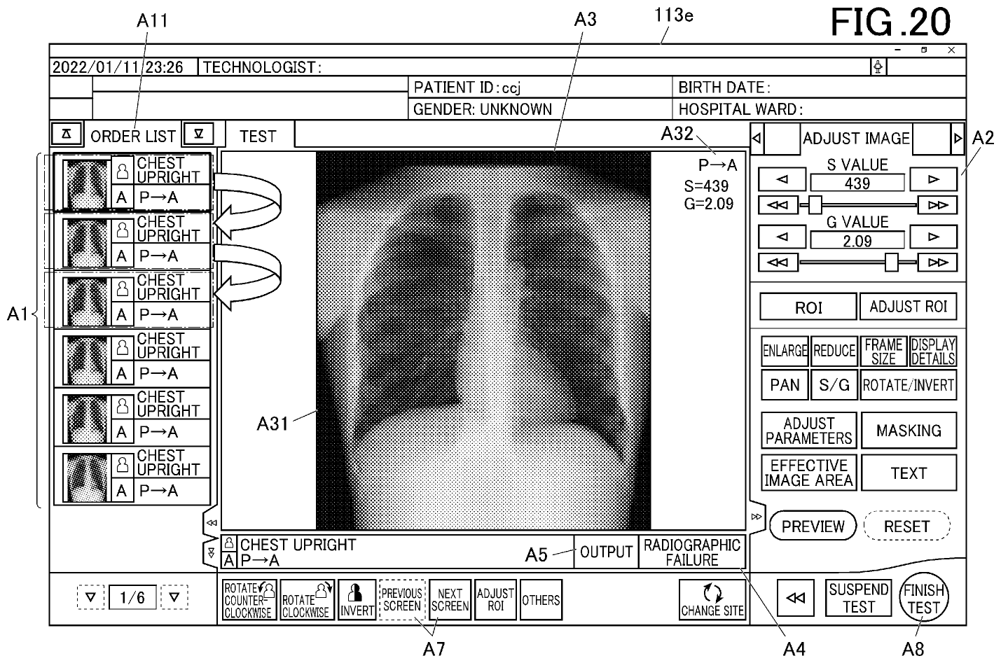

In a radiographing process, first, the controller 111 of the radiography control device 11 displays, on the display 113, a test screen 113e shown in FIG. 20, and accepts selection of the test order for execution of radiographing by the radiographer (Step S31). The radiographer selects the test order for execution of radiographing from among registered test orders by pressing a radiographing selection button A1.

FIG. 20 shows an example of the test screen 113e displayed on the display 113.

The test screen 113e is provided with not only the radiographing selection buttons A1 where each piece of content (the radiography site, radiographing direction, etc.) included in the test order information, but also a setting area A2 for image adjustment for selected radiographing, an image display area A3 for displaying a taken image, a radiographic failure button A4, an image output button A5 for outputting an image to the external apparatus, a switching button A7 (first operation receiver) for switching the image to be displayed in the image display area A3, a test finish button A8, and the like. Note that at the stage of Step S31, no image is displayed in the image display area A3.

When the test order is selected, the controller 111 transmits the radiographing condition included in the test order to the X-ray generating control device 21 and the FPD 12, and configures the setting (Step S32).

During this, the radiographer arranges the test subject between the X-ray tube device 25 and the radiographic stand 13, thus achieving positioning. Here, the positioning is a setting of the body posture of the test subject during radiographing, for example. The radiographer instructs the test subject to take a certain respiratory status (deep respiration, etc.).

Next, the controller 111 accepts a press operation on the irradiation switch 23 by the radiographer, and performs radiographing (Step S33).

In a case where an irradiation time period is set as in simple X-ray radiography, irradiation with X-rays is finished upon a lapse of the predetermined irradiation time period. In the case of dynamic radiographing, irradiation with X-rays is continuously performed in a time period in which the second-step press operation on the irradiation switch 23 is applied, and when the press operation on the irradiation switch 23 is released, the irradiation with X-rays is finished.

The taken image is transmitted from the FPD 12 to the radiography control device 11.

Here, the radiographer is assumed to be a state of having performed the radiographing multiple times. That is, the controller 111 obtains a plurality of radiographs taken by the same radiographer (radiographs satisfying the predetermined condition). Note that the plurality of radiographs satisfying the predetermined condition may be a plurality of radiographs in accordance with the test order, a plurality of radiographs taken by radiographing the same test subject, a plurality of radiographs taken by radiographing on the same radiography date, a plurality of radiographs taken by radiographing the same radiography site, or a plurality of radiographs taken by radiographing in a predetermined time period. The plurality of radiographs satisfying the predetermined condition are only required to be a plurality of radiographs taken in one test. Across the plurality of radiographs, the radiographers, the radiography sites, and the medical departments may vary. Across the plurality of radiographs, a plurality of conditions among the same test subject, the same radiography date, the same radiography site, the same radiographer, and radiographing in the predetermined time period may be satisfied. The plurality of radiographs satisfying the predetermined condition may be a plurality of frame images taken by one time of dynamic radiographing. The plurality of radiographs satisfying the predetermined condition may be a plurality of frame images taken by multiple times of dynamic radiographing in one test.

As shown in FIG. 20, the controller 111 then displays a taken image A31, and the radiographing condition A32 in the radiographing, in the image display area A3.

Next, the controller 111 accepts determination of whether or not the image displayed in the image display area A3 is regarded as a radiographic failure by the radiographer (radiographic failure determination) (Step S34). To regard it as a radiographic failure, the radiographer presses the radiographic failure button A4.

After the radiographic failure determination is finished, the radiographer releases the test subject from the state of being positioned.

Next, the controller 111 accepts an instruction by the user for execution of the post-processes to the image through the operation receiver 114, executes the post-processes (Step S35), and transfers the processing to Step S31, thus allowing transition to next radiographing. Here, the user may be a technologist, a radiologist, a diagnostician or the like, other than the radiographer.

The post-processes include at least one of ROI (Region Of Interest) adjustment, effective image area setting, S/G value adjustment, rotation/inversion/rotate by any angle, image processing (E process/F process/H process/scattered radiation correction/image processing condition changing, etc.), emphasizing processes (a catheter distal end emphasizing process, a gauze emphasizing process, and/or other emphasizing processes, etc., as frequency emphasizing processes), a grid removal process, masking, trimming, marker/stump/overlay, and output setting.

In Step S35, the controller 111 stores, in the storage 112, a result of execution (presence or absence of execution and execution content) of the post-processes to the image (Switching process) Next, a switching process executed by the controller 111 in Step S35 described above is described.

The switching process is a process for switching the image displayed in the image display area A3, based on an input operation by the user through the operation receiver 114. Here, the user may be a technologist, a radiologist, a diagnostician or the like, other than the radiographer.

Specifically, if an instruction for executing a specific process for the image displayed in the image display area A3 is issued by the user, the controller 111 designates the specific process as the last process among the post-processes, and in this case, switches the display displayed in the image display area A3 to the next image Thus, the operation of selecting the next image by the user can be reduced.

Switching of the image displayed in the image display area A3 to the next image means switching to an image of the next test order displayed in a test order list A11 shown in FIG. 20 (an image taken by radiography corresponding to the next radiography selection button A1). In a case where one time of radiography corresponding to the radiography selection button A1 includes a plurality of images, switching may mean switching to the next image among the plurality of images. The radiography selection button A1 that is a switching destination is not limited to a button for radiography that has already been radiographed (a radiography selection button A1 including a taken image), and may be a radiography selection button A1 for radiography that has not been executed yet and scheduled to be radiographed. The radiography selection button A1 that is a switching destination may be preset from among any of the next radiography selection button A1 in the sequence in the test order list A11, any of the radiography selection buttons A1 for radiography that has been executed, and any of the radiography selection buttons A1 for radiography that has not been executed yet.

For example, in the post-processes, the image output button A5 is highly possibly pressed lastly. Accordingly, when the image output button A5 is pressed by the user, the controller 111 switches the image displayed in the image display area A3 to the next image That is, in this case, the image output button A5 functions as a second operation receiver that has a function different from that of the switching button A7 (first operation receiver). When the processor switches multiple radiographs and executes the post-processes (predetermined processes), the controller 111 assigns the image output button A5 (second operation receiver) a function of switching the radiograph to be displayed on the display 113.

For example, if the user issues an instruction for executing the catheter distal end emphasizing process, the gauze emphasizing process, masking or the like, as the post-processes, to the image displayed in the image display area A3, the controller 111 may switch the image to be displayed in the image display area A3 to the next image after displaying each emphasizing-processed image for a predetermined time period (for example, after several seconds). These emphasizing processes is often used for status confirmation after a surgical operation. Only if the emphasizing-processed image is successfully confirmed, the purpose of the taken image is accomplished. Accordingly, the controller 111 determines that the emphasizing processes are the final processes.

For example, if the user issues an instruction for executing ROI adjustment, as the post-process, to the image displayed in the image display area A3, the controller 111 may switch the image to be displayed in the image display area A3 to the next image after a predetermined time period since the ROI adjustment has been executed (for example, after several seconds).

For example, if the user issues an instruction for full-screen display of the image displayed in the image display area A3 in the post-processes, the controller 111 may switch the image to be displayed in the image display area A3 to the next image after a predetermined time period since the full-screen display has been executed (for example, after several seconds). It is because, for example, if an image is displayed on a portable terminal used for rounds and the like, the portable terminal has a small screen, and in some cases, the post-processed image is enlarged in full-screen display and confirmed, and the post-processes are then finished if no problem is found. In such cases, switching may be made to the next image if there is no input (touch operation) for several seconds on the portable terminal after execution of full-screen display.

That is, in this case, the button in the test screen 113e for issuing an instruction for execution of the catheter distal end emphasizing process, the gauze emphasizing process, masking, or ROI adjustment, or full-screen display, as the post-process, is a second operation receiver that has a function different from that of the switching button A7 (first operation receiver), and is for receiving an input of completion of the post-processes (predetermined processes) by the processor.

Note that the button functioning as the second operation receiver may be set with respect to each radiography site, each user, each test type, each image type, each medical department, or the like. Here, the user may be a technologist, a radiologist, a diagnostician, or the like, other than the radiographer.

As described above, by executing the switching process, the next image selection operation by the user can be reduced, and therefore the predetermined processes (post-processes) can be more efficiently executed to the plurality of radiographs.

Note that in Step S35 in the radiographing process described above, if, for example, an instruction for executing the gradation process as the post-process for the image displayed in the image display area A3 is issued by the user, since there is a low possibility that the frame of the effective image area is subsequently changed, the controller 111 may set the frame of the effective image area at the time as the trimming frame in order to reduce the number of operations by the user.

### (Modification)

Next, a modification of the third embodiment is described. Hereinafter, points different from those of the first embodiment are mainly described.

In this modification, the operation receiver 114 functions as a third operation receiver operable by the user. Here, the user may be a technologist, a radiologist, a diagnostician, or the like, other than the radiographer.

The controller 111 determines whether the post-processes (predetermined processes) have been completed based on an operation performed by the user on the operation receiver 114 (third operation receiver). Here, the controller 111 functions as a determiner.

When the processor switches multiple radiographs and executes the post-processes (predetermined processes) and the determiner determines that the post-processes (predetermined processes) have been completed, the controller 111 switches the radiograph displayed on the display 113.

In the switching process in this modification, when a specific operation is performed by the user in the test screen 113e, the controller 111 determines that the post-processes to the image currently displayed in the image display area A3 have been completed. In this case, the controller 111 switches the image displayed in the image display area A3 to the next image.

For example, when the user double-clicks an area where the image A31 is not displayed in the image display area A3, or an area out of a trimming frame, the controller 111 determines that the post-processes to the image currently displayed in the image display area A3 have been completed. The controller 111 then switches the image displayed in the image display area A3 to the next image.

For example, when the user drags and drops the trimming frame in the image display area A3, the controller 111 determines that the post-processes to the image currently displayed in the image display area A3 have been completed. The controller 111 may then switch the image displayed in the image display area A3 to the next image after a predetermined time period since the trimming frame was dropped (for example, after several seconds).

### (Other)

Note that, in Step S35 of the radiographing process described above, it is preferable that by limiting the user's operation range in the test screen 113e, the operation load on the user is reduced.

For example, the controller 111 may display an enlargement button and a reduction button in the setting area A2 as one button in the test screen 113e, and switch between enlargement and reduction by a pressing place in the button or a pressing method (for example, double click or long press). Without any limitation only to the enlargement button and the reduction button, the controller 111 may display functions (buttons) that are close to each other or related to each other as one button, and switch the functions to be performed by the pressing place in the button or the pressing method.

Accordingly, the range of operation by the user in the test screen 113e for issuing an instruction for executing the post-processes can be narrowed, or the operation portion can be increased in size, which can facilitate pressing, and reduce the operation load on the user.

### Fourth Embodiment

Next, an X-ray radiographic system 1 according to a fourth embodiment is described. Hereinafter, points different from those of the third embodiment are mainly described.

In this embodiment, when the controller 111 executes the post-processes (predetermined processes) to a first radiograph, and subsequently executes the post-processes (predetermined processes) to a second radiograph, the controller 111 determines the display mode of the operation receiver displayed on the display 113 for executing the post-processes (predetermined processes) for the second radiograph, in accordance with the post-processes (predetermined processes) executed to the first radiograph. Here, the controller 111 functions as a display controller.

### (Display Control Process)

Next, in Step S35 of the radiographing process in this embodiment, a display control process executed by the controller 111 is described.

As for the display control process, when the controller 111 switches the image displayed in the image display area A3 to the next image, the controller 111 maintains the display mode for executing a specific process, and reduces a selection operation by the user for executing the process again. Here, the user may be a technologist, a radiologist, a diagnostician, or the like, other than the radiographer.

The specific process is the last process applied to the image displayed in the image display area A3, a preset process, or the like.

The specific process may be set in accordance with the radiography site and the radiographing condition.

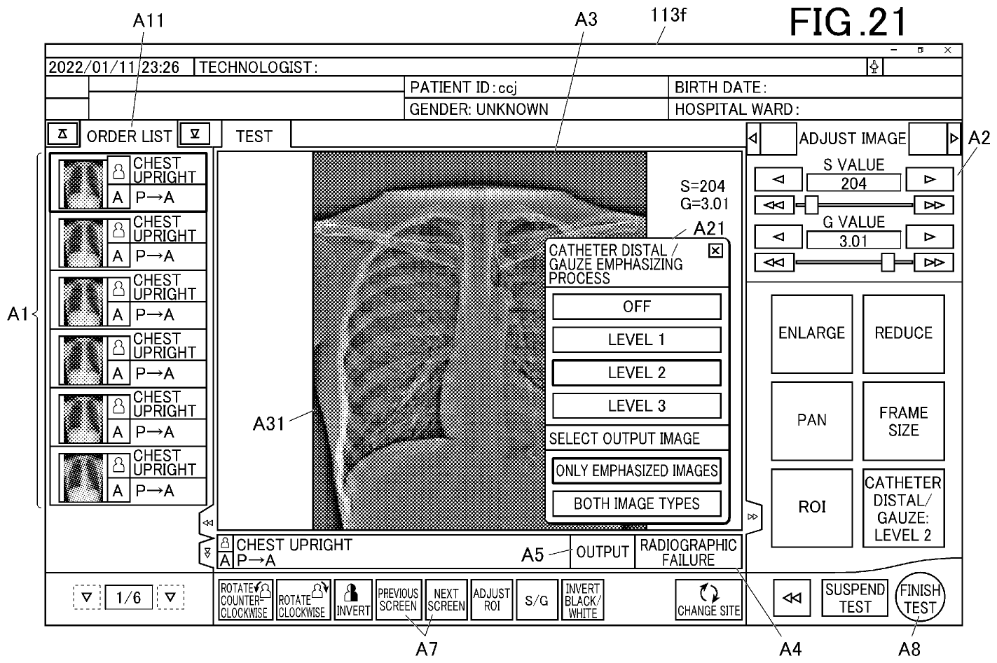

For example, if an instruction for executing the specific process for the image displayed in the image display area A3 is issued by the user through a subpanel A21 (see FIG. 21), the controller 111 displays the subpanel A21 in a test screen 113f upon switching of the image displayed in the image display area A3 to the next image. The subpanel A21 is a screen displayed by the controller 111 when a predetermined button in the setting area A2 is pressed by the user.

That is, when the controller 111 executes the post-processes for a first radiograph, and subsequently executes the post-processes for a second radiograph (the next image of the first radiograph), the controller 111 determines and displays the display mode of the operation receiver displayed on the display 113 for executing the post-processes to the second radiograph, in accordance with the post-processes executed to the first radiograph. In this case, when the controller 111 executes the post-processes to the second radiograph, the controller 111 determines to maintain the display mode of the operation receiver (a state where the subpanel A21 is displayed) during execution of the post-processes to the first radiograph. That is, the operation receiver includes buttons (first button group) in the setting area A2, and the subpanel A21 (second button group) displayed by the controller 111 (display controller) when the first button group is pressed. When the controller 111 (display controller) displays the second button group during execution of the post-processes (predetermined processes) to the first radiograph, the controller 111 determines to display the second button group during execution of the post-processes (predetermined processes) to the second radiograph.

The controller 111 may perform display of a specific process in a dialog display, a tree structure, or the like, besides or instead of the subpanel A21.

For example, when the controller 111 executes the catheter distal end emphasizing process and the gauze emphasizing process as the post-processes, the controller 111 displays a dialog for changing the emphasizing level of the emphasizing process in the test screen 113f upon switching of the image displayed in the image display area A3 to the next image.

For example, when the setting area A2 includes a hierarchic structure or a tree structure, and the user issues an instruction for executing the specific process through the setting area A2, the controller 111 may display the hierarchic structure or the tree structure in the setting area A2 in an open state upon switching of the image displayed in the image display area A3 to the next image.

The controller 111 may perform the display control in the display control process in accordance with the radiography situation (test order type).

For example, a case where the catheter distal end emphasizing process and the gauze emphasizing process are executed as the post-processes is described. In this case, if the next test order has not been radiographed when the image displayed in the image display area A3 is switched to the image of the next test order, the controller 111 does not display a dialog for changing the emphasizing level of the emphasizing process. If the next test order is in a radiographed state immediately after radiography (at the time of radiographic failure determination), the dialog is displayed in the test screen 113f. If the next test order is in the radiographed state during execution of the post-processes, the dialog is not displayed, for example. That is, in accordance with the radiography situation, the controller 111 displays the display mode of the operation receiver displayed on the display 113.

The controller 111 may determine the display mode of the operation receiver displayed on the display 113, based on at least any of the user, test type, image type, and medical department, and perform display control in the display control process described above. Here, the user may be a technologist, a radiologist, a diagnostician, or the like, other than the radiographer.

The controller 111 may perform display control in the test order list A11, as the display control process, in accordance with the post-process execution result (presence or absence of execution and execution content). That is, the controller 111 determines the display order of the test order (radiographic order) on the display 113 in accordance with the post-process execution state.

For example, the controller 111 displays the radiography selection buttons A1 in the order of image outputting from the top, or in the order of executing the post-processes, in the test order list A11.

For example, the controller 111 may display the radiography selection buttons A1 in the order of executing radiography from the top, comprehensively display the buttons A1 for the same radiography site, or display buttons A1 what have been subjected to no radiographic failure determination at higher orders on a priority basis, in the test order list A11.

For example, in a case where the controller 111 has a function of determining whether or not the post-processes are required to be executed for the taken image, the controller 111 may display the radiography selection buttons A1 associated respectively with the images required to be subjected to the post-processes at higher orders on a priority basis, in the test order list A11.

To execute the same specific processes, as post-processes, to a plurality of images, the controller 111 may display the radiography selection buttons A1 associated respectively with the target images of the specific process at higher orders on a priority basis, in the test order list A11.

For example, to assign the irradiation direction and the like to a plurality of images in a similar overlaid manner, the radiography selection buttons A1 associated respectively with the assignment target images may be displayed at higher orders on a priority basis.

The controller 111 may display the radiography selection buttons A1 with respect to each user and each facility in a preset order in the test order list A11.

As described above, the controller 111 switches the image displayed in the image display area A3 to the next image in the switching process described above, in an order displayed from the top on the test order list A11.

As described above, the radiography control device 11 (control device) includes: the obtainer (controller 111) that obtains a plurality of radiographs satisfying the predetermined condition; the processor (controller 111) that executes the predetermined processes (post-processes) to the plurality of radiographs obtained by the obtainer; the display 113 that displays the radiograph as a target to which the predetermined process is executed by the processor; the first operation receiver (switching button A7) for switching the radiograph displayed on the display 113; the second operation receiver (e.g., the image output button A5) that has a function different from that of the first operation receiver; and the controller 111 that assigns the second operation receiver a function of switching the radiograph displayed on the display 113 when the plurality of radiographs are switched and subjected to the predetermined processes by the processor.

Consequently, since the next image selection operation by the user can be reduced, the predetermined processes (post-processes) can be more efficiently executed to the plurality of radiographs.

The radiography control device 11 includes: the obtainer (controller 111) that obtains a plurality of radiographs satisfying the predetermined condition; the processor (controller 111) that executes the predetermined processes (post-processes) to the plurality of radiographs obtained by the obtainer; the display 113 that displays the radiograph as a target to which the predetermined processes are executed by the processor; the third operation receiver (operation receiver 114) operable by the user; the determiner (controller 111) that determines whether the predetermined processes are completed based on an operation performed by the user on the third operation receiver; and the controller 111 that switches the radiograph displayed on the display 113 when the plurality of radiographs are switched and subjected to the predetermined processes by the processor and it is determined by the determiner that the predetermined processes are completed.

Consequently, since the next image selection operation by the user can be reduced, the predetermined processes (post-processes) can be more efficiently executed to the plurality of radiographs.

In the radiography control device 11, the predetermined condition is any of a radiographic target being an identical test subject, an identical radiography date, an identical radiography site, and an identical user.

Consequently, for example, even in a case where a large number of radiographing operations are performed a day, since the next image selection operation by the user can be reduced, the predetermined processes can be more efficiently executed to the plurality of radiographs.

In the radiography control device 11, the second operation receiver is the image output button A5 for outputting, to the external apparatus, the radiograph to which the predetermined processes have been executed by the processor.

Consequently, based on pressing of the image output button A5 having a high possibility of being pressed lastly in the post-processes, the radiograph displayed on the display 113 can be appropriately switched.

In the radiography control device 11, the second operation receiver is a button for inputting completion of the predetermined processes by the processor.

Consequently, based on pressing of the button for inputting completion of the post-processes, the radiograph displayed on the display 113 can be appropriately switched.

In the radiography control device 11, the second operation receiver is preset with respect to each user, each test type, each image type, or each medical department.

Consequently, the radiograph to be displayed on the display 113 can be switched at timing suitable for the user, test type, the image type, or the medical department.

In the radiography control device 11, the plurality of radiographs include a plurality of frame images.

Consequently, also in dynamic radiographing, since the next image selection operation by the user can be reduced, the predetermined processes (post-processes) can be more efficiently executed to the plurality of radiographs.

Although the embodiments of the present invention have been described, the scope of the present invention is not limited to the embodiments described above, and encompasses the scope of the invention described in the claims and their equivalent range.

For example, in the embodiment described above, the X-ray radiographic system 1 is installed in the radiographing room. There is no limitation to this. The X-ray radiographic system 1 may be configured as a visiting car, which is movable.

Although embodiments of the present invention have been described and illustrated in detail, the disclosed embodiments are made for purposes of illustration and example only and not limitation. The scope of the present invention should be interpreted by terms of the appended claims.

1. A control device, comprising a hardware processor that obtains a plurality of radiographs satisfying a predetermined condition, and
identifies a radiograph to be subjected to a predetermined process, from among the plurality of radiographs obtained.

2. The control device according to claim 1, wherein the predetermined condition is any of a radiographic target being an identical test subject, an identical radiography date, an identical radiography site, and an identical user.

3. The control device according to claim 1, wherein the hardware processor identifies the radiograph to be subjected to the predetermined process, based on information about an image including a previously radiographed identical radiography site.

4. The control device according to claim 1, wherein the hardware processor identifies an image corresponding to at least any of a predetermined radiography site, predetermined usage, and a respiratory cycle, as the radiograph to be subjected to the predetermined process.

5. The control device according to claim 1, wherein the hardware processor identifies the radiograph to be subjected to the predetermined process, based on an instruction by a user.

6. The control device according to claim 1, wherein the hardware processor identifies the radiograph to be subjected to the predetermined process, based on external information.

7. The control device according to claim 1, wherein the hardware processor presents a display for discriminating the identified radiograph to be subjected to the predetermined process from an unidentified radiograph.

8. The control device according to claim 7, wherein the hardware processor does not display the unidentified radiograph during execution of the predetermined process.

9. The control device according to claim 1, wherein the hardware processor executes the predetermined process, to the identified radiograph to be subjected to the predetermined process.

10. The control device according to claim 1, wherein the hardware processor accepts execution of the predetermined process, by a user, for the identified radiograph to be subjected to the predetermined process.

11. The control device according to claim 1, wherein the plurality of radiographs include a plurality of frame images.

12. A control method, comprising:
obtaining a plurality of radiographs satisfying a predetermined condition; and
identifying a radiograph to be subjected to a predetermined process, from among the plurality of radiographs obtained by the obtaining.

13. A non-transitory computer readable recording medium storing a program causing a computer used for a control device to perform:
obtaining a plurality of radiographs satisfying a predetermined condition, and
identifying a radiograph to be subjected to a predetermined process, from among the plurality of radiographs obtained.
